(19) United States
(12) Patent Application Publication
(10) Pub. No.: US 2019/0350819 A1
(43) Pub. Date: Nov. 21, 2019

(54) CONDITIONING HAIR COMPOSITIONS IN THE FORM OF DISSOLVABLE SOLID STRUCTURES

(71) Applicant: The Procter & Gamble Company, Cincinnati, OH (US)

(72) Inventors: Mark William Hamersky, Hamilton, OH (US); Jennifer Elaine Hilvert, Cincinnati, OH (US); Howard David Hutton, III, Oregonia, OH (US); Min Mao, Deerfield Township, OH (US)

(21) Appl. No.: 15/981,096

(22) Filed: May 16, 2018

(51) Int. Cl. A61K 8/02; A61K 8/81; A61K 8/34; A61K 8/46; A61K 8/41; A61Q 5/12

(52) U.S. Cl. CPC A61K 8/027; A61K 8/8129; A61Q 5/12; A61K 8/463; A61K 8/416; A61K 8/342

(57) ABSTRACT

A dissolvable solid structure that can contain heterogeneous filaments. The filaments can contain a water-soluble polymeric structurant and dehydrated gel network globules containing fatty alcohols and cationic surfactants.

### FIELD OF THE INVENTION

The present invention relates to conditioning hair care compositions in the form of dissolvable solid structures. The dissolvable solid structures comprise a polymeric structurant, a fatty amphiphiles and a cationic surfactant.

### BACKGROUND OF THE INVENTION

Many personal care and other consumer products in the market today are sold in liquid form. While widely used, liquid products often have tradeoffs in terms of packaging, storage, transportation, and convenience of use. Liquid consumer products typically are sold in bottles which add cost as well as packaging waste, much of which ends up in land-fills.

Hair Care products in the form of a dissolvable solid structures present an attractive form to consumers. Market executions of dissolvable solid structures may include, dissolvable films, compressed powders in a solid, fibrous structures, porous foams, soluble deformable solids, powders, etc. However, many of these executions have consumer negatives during in use experience. For example, these products typically do not provide sufficient wet and dry conditioning to the hair. Products such as bars or prills, do not hydrate fast enough in the shower to satisfy the consumer's desire to quickly apply to the hair without undue effort to dissolve the product.

A need therefore still exists for dissolvable solid structures which deliver the desired wet and dry conditioning to the hair, and to improve the dissolving properties of the solid product to facilitate improved consumer in use satisfaction. A need also exists for a dissolvable solid structure that is not in a lamellar state when dry, yet yields a lamellar state upon wetting.

### SUMMARY OF THE INVENTION

A dissolvable solid structure comprising filaments wherein the filaments comprise: (a) from about 30% to about 45%, by weight on a dry filament basis, of a water-soluble polymeric structurant; (b) from about 15% to about 27%, by weight on a dry filament basis, of a fatty alcohol having a carbon chain length from about 16 to about 22 carbon atoms or mixtures thereof and a melting point above 25° C.; (c) from about 5% to about 10%, by weight on a dry filament basis, cationic surfactant; wherein the filament is heterogeneous upon microscopic inspection and comprises one or more globules comprising a dehydrated gel network wherein the dehydrated gel network comprises the fatty alcohol and the cationic surfactant.

A dissolvable solid structure comprising filaments wherein the filaments comprise: (a) from about 15% to about 55%, by weight on a dry filament basis, of a polymeric structurant; (b) a globule comprising a dehydrated gel network comprising a high melting point fatty material having a carbon chain length C12-C22 or mixtures thereof, wherein the melting point is above 25° C.; and a cationic surfactant; wherein the filaments are heterogeneous upon microscopic inspection; wherein the dissolvable structure comprises a lamellar structure upon addition of water to the dissolvable solid structure in the ratio of about 5:1.

A dissolvable solid structure comprising filaments wherein the filaments comprise: (a) from about 30% to about 45%, by weight on a dry filament basis, of a water-soluble polymer selected from the group consisting of polyvinyl alcohol, vinyl acetate-vinyl alcohol copolymer, and combinations thereof; (b) from about 15% to about 27%, by weight on a dry filament basis, of a fatty alcohol selected from the group consisting of fatty alcohol is selected from the group consisting of cetyl alcohol, stearyl alcohol, behenyl alcohol, and combinations thereof; (c) from about 5% to about 10%, by weight on a dry filament basis, of a mono-long alkyl quaternized ammonium salt selected from the group consisting of behenyl trimethyl ammonium salt; stearyl trimethyl ammonium salt; cetyl trimethyl ammonium salt; and hydrogenated tallow alkyl trimethyl ammonium salt, and combinations thereof; wherein the filament is heterogeneous upon microscopic inspection and comprises one or more globules comprising a dehydrated gel network wherein the dehydrated gel network comprises the fatty alcohol and the cationic surfactant; wherein the dissolvable solid structure is a hair conditioner.

### BRIEF DESCRIPTION OF THE DRAWINGS

FIG. 1 is a schematic representation of an example of a fibrous element, in this case a filament, according to the present invention;

FIG. 2 is a schematic representation of an example of a fibrous structure comprising a plurality of filaments according to the present invention;

FIG. 3 is a scanning electron microscope photograph of a cross-sectional view of an example of a fibrous structure according to the present invention;

FIG. 4 is a schematic representation of a cross-sectional view of another example of a fibrous structure according to the present invention;

FIG. 5 is a schematic representation of a cross-sectional view of another example of a fibrous structure according to the present invention;

FIG. 6 is a scanning electron microscope photograph of a cross-sectional view of another example of a fibrous structure according to the present invention;

FIG. 7 is a schematic representation of a cross-sectional view of another example of a fibrous structure according to the present invention;

FIG. 8 is a schematic representation of a cross-sectional view of another example of a fibrous structure according to the present invention;

FIG. 9 is a schematic representation of a cross-sectional view of another example of a fibrous structure according to the present invention;

FIG. 10 is a schematic representation of a cross-sectional view of another example of a fibrous structure according to the present invention;

FIG. 11 is a schematic representation of an example of a process for making an example of a fibrous structure according to the present invention;

FIG. 12 is a schematic representation of an example of a die with a magnified view used in the process of FIG. 11;

FIGS. 13A and B is a light microscopy image of a filament at 100× magnification;

FIG. 14 is a schematic representation of an example of another process for making an example of a fibrous structure according to the present invention;

FIG. 15 is a schematic representation of another example of a process for making another example of a fibrous structure according to the present invention;

FIG. 16 is a schematic representation of another example of a process for making another example of a fibrous structure according to the present invention; and

FIG. 17 is a representative image of an example of a patterned belt useful in the processes for making the fibrous structure according to the present invention.

### DETAILED DESCRIPTION OF THE INVENTION

### A. Definitions

As used herein, The Dissolvable Solid Structure may be referred to herein as “the Dissolvable Solid Structure”, “the Structure”, or “the Dissolvable Structure”.

As used herein, “dissolvable” means that the Dissolvable Solid Structure is completely soluble in water or it provides a uniform dispersion upon mixing in water according to the hand dissolution test. The Dissolvable Solid Structure has a hand dissolution value of from about 1 to about 30 strokes, alternatively from about 2 to about 25 strokes, alternatively from about 3 to about 20 strokes, and alternatively from about 4 to about 15 strokes, as measured by the Hand Dissolution Method.

As used herein, “flexible” means a Dissolvable Solid Structure meets the distance to maximum force values discussed herein.

“Fibrous structure” as used herein means a structure that comprises one or more fibrous elements and optionally, one or more particles. The fibrous structure as described herein can mean an association of fibrous elements and optionally, particles that together form a structure, such as a unitary structure, capable of performing a function.

The fibrous structures of the present invention may be homogeneous or may be layered. If layered, the fibrous structures may comprise at least two and/or at least three and/or at least four and/or at least five layers, for example one or more fibrous element layers, one or more particle layers and/or one or more fibrous element/particle mixture layers. A layer may comprise a particle layer within the fibrous structure or between fibrous element layers within a fibrous structure. A layer comprising fibrous elements may sometimes be referred to as a ply. A ply may be a fibrous structure which may be homogeneous or layered as described herein.

In one example, a single-ply fibrous structure according to the present invention or a multi-ply fibrous structure comprising one or more fibrous structure plies according to the present invention may exhibit a basis weight of less than 5000 g/m² as measured according to the Basis Weight Test Method described herein. In one example, the single- or multi-ply fibrous structure according to the present invention may exhibit a basis weight of greater than 10 g/m² to about 5000 g/m² and/or greater than 10 g/m² to about 3000 g/m² and/or greater than 10 g/m² to about 2000 g/m² and/or greater than 10 g/m² to about 1000 g/m² and/or greater than 20 g/m² to about 800 g/m² and/or greater than 30 g/m² to about 600 g/m² and/or greater than 50 g/m² to about 500 g/m² and/or greater than 300 g/m² to about 3000 g/m² and/or greater than 500 g/m² to about 2000 g/m² as measured according to the Basis Weight Test Method.

In one example, the fibrous structure of the present invention is a “unitary fibrous structure.”

“Unitary fibrous structure” as used herein is an arrangement comprising a plurality of two or more and/or three or more fibrous elements that are inter-entangled or otherwise associated with one another to form a fibrous structure and/or fibrous structure plies. A unitary fibrous structure of the present invention may be one or more plies within a multi-ply fibrous structure. In one example, a unitary fibrous structure of the present invention may comprise three or more different fibrous elements. In another example, a unitary fibrous structure of the present invention may comprise two or more different fibrous elements.

“Article” as used herein refers to a consumer use unit, a consumer unit dose unit, a consumer use saleable unit, a single dose unit, or other use form comprising a unitary fibrous structure and/or comprising one or more fibrous structures of the present invention.

“By weight on a dry filament basis” means that the weight of the filament measured immediately after the filament has been conditioned in a conditioned room at a temperature 73° F.±4° F. (about 23° C.±2.2° C.) and a relative humidity of 50%±10% for 2 hours. Similarly, “by weight on a dry fibrous element basis” or “by weight on a dry fibrous structure basis” means the weight of the fibrous element or structure after the fibrous element has been conditioned in a conditioned room at a temperature of 73° F.±4° F. (about 23° C.±2.2° C.) and a relative humidity of 50%±10% for 2 hours.

“Fibrous element” as used herein means an elongate particulate having a length greatly exceeding its average diameter, i.e. a length to average diameter ratio of at least about 10. A fibrous element may be a filament or a fiber. In one example, the fibrous element is a single fibrous element rather than a yarn comprising a plurality of fibrous elements.

The fibrous elements of the present invention may be spun from a filament-forming compositions also referred to as fibrous element-forming compositions via suitable spinning process operations, such as meltblowing, spunbonding, electro-spinning, and/or rotary spinning.

The fibrous elements of the present invention may be monocomponent (single, unitary solid piece rather than two different parts, like a core/sheath bicomponent) and/or multicomponent. For example, the fibrous elements may comprise bicomponent fibers and/or filaments. The bicomponent fibers and/or filaments may be in any form, such as side-by-side, core and sheath, islands-in-the-sea and the like.

“Filament” as used herein means an elongate particulate as described above that exhibits a length of greater than or equal to 5.08 cm (2 in.) and/or greater than or equal to 7.62 cm (3 in.) and/or greater than or equal to 10.16 cm (4 in.) and/or greater than or equal to 15.24 cm (6 in.).

Filaments are typically considered continuous or substantially continuous in nature. Filaments are relatively longer than fibers. Non-limiting examples of filaments include meltblown and/or spunbond filaments.

“Fiber” as used herein means an elongate particulate as described above that exhibits a length of less than 5.08 cm (2 in.) and/or less than 3.81 cm (1.5 in.) and/or less than 2.54 cm (1 in.).

Fibers are typically considered discontinuous in nature. Non-limiting examples of fibers include staple fibers produced by spinning a filament or filament tow of the present invention and then cutting the filament or filament tow into segments of less than 5.08 cm (2 in.) thus producing fibers.

In one example, one or more fibers may be formed from a filament of the present invention, such as when the filaments are cut to shorter lengths (such as less than 5.08 cm in length). Thus, in one example, the present invention also includes a fiber made from a filament of the present invention, such as a fiber comprising one or more filament-forming materials and one or more additives, such as active agents. Therefore, references to filament and/or filaments of the present invention herein also include fibers made from such filament and/or filaments unless otherwise noted. Fibers are typically considered discontinuous in nature relative to filaments, which are considered continuous in nature.

“Filament-forming composition” and/or “fibrous element-forming composition” as used herein means a composition that is suitable for making a fibrous element of the present invention such as by meltblowing and/or spunbonding. The filament-forming composition comprises one or more filament-forming materials that exhibit properties that make them suitable for spinning into a fibrous element. In one example, the filament-forming material comprises a polymer. In addition to one or more filament-forming materials, the filament-forming composition may comprise one or more additives, for example one or more active agents, such as a conditioning agent. In addition, the filament-forming composition may comprise one or more polar solvents, such as water, into which one or more, for example all, of the filament-forming materials and/or one or more, for example all, of the active agents are dissolved and/or dispersed prior to spinning a fibrous element, such as a filament from the filament-forming composition.

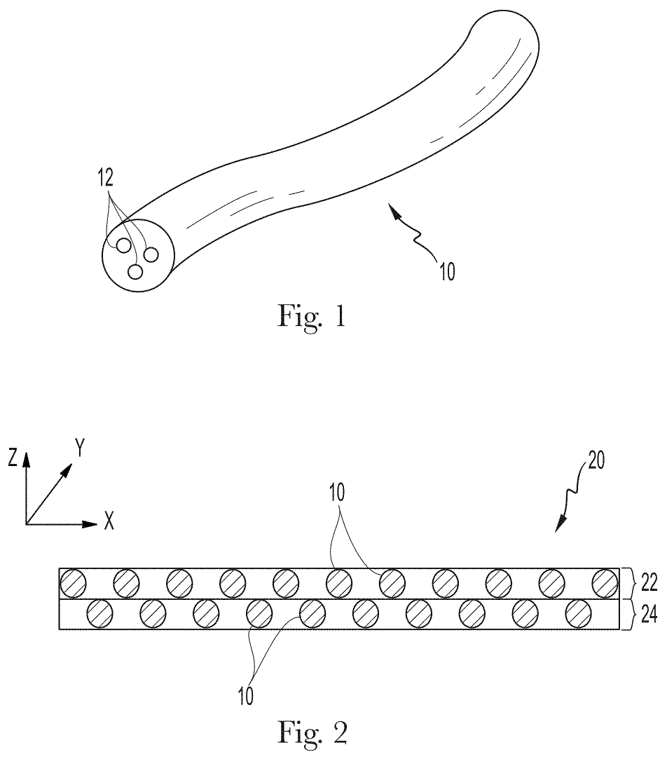

For example, as shown in FIG. 1, a fibrous element, such as a filament 10 made from a fibrous element-forming composition such that one or more additives 12, for example one or more active agents, may be present in the filament rather than on the filament, such as a coating composition comprising one or more active agents, which may be the same or different from the active agents in the fibrous elements and/or particles.

In one example, one or more additives, such as active agents, may be present in the fibrous element and one or more additional additives, such as active agents, may be present on a surface of the fibrous element. In another example, a fibrous element of the present invention may comprise one or more additives, such as active agents, that are present in the fibrous element when originally made, but then bloom to a surface of the fibrous element prior to and/or when exposed to conditions of intended use of the fibrous element.

As shown in FIG. 2, an example of a dissolvable solid structure 20 of the present invention, for example a multi-ply fibrous structure according to the present invention may comprise two or more different fibrous structure layers or plies 22, 24 (in the z-direction of the dissolvable solid structure 20 of filaments 10 of the present invention that form the fibrous structures of the dissolvable solid structure 20. The filaments 10 in layer 22 may be the same as or different from the filaments 10 in layer 24. Each layer or ply 22, 24 may comprise a plurality of identical or substantially identical or different filaments. For example, filaments that may release their active agents at a faster rate than others within the dissolvable solid structure 20 and/or one or more fibrous structure layers or plies 22, 24 of the dissolvable solid structure 20 may be positioned as an external surface of the dissolvable solid structure 20. The layers or plies 22 and 24 may be associated with each other by mechanical entanglement at their interface between the two layers or plies and/or by thermal or adhesive bonding and/or by depositing one of the layers or plies onto the other existing layer or ply, for example spinning the fibrous elements of layer or ply 22 onto the surface of the layer or ply 24.

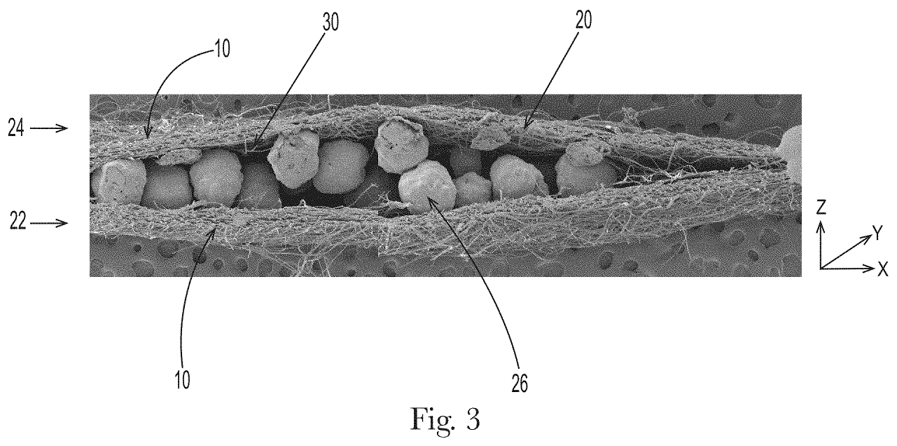

As shown in FIG. 3, another example of an dissolvable solid structure 20, for example a fibrous structure according to the present invention comprises a first fibrous structure layer or ply 22 comprising a plurality of fibrous elements, for example filaments 10, a second fibrous structure layer 24 comprising a plurality of fibrous elements, for example filaments 10, and a plurality of particles or a particle layer 26 positioned between the first and second fibrous structure layers 22 and 24. A similar fibrous structure can be formed by depositing a plurality of particles on a surface of a first ply of fibrous structure comprising a plurality of fibrous elements and then associating a second ply of fibrous structure comprising a plurality of fibrous elements such that the particles or a particle layer are positioned between the first and second fibrous structure plies.

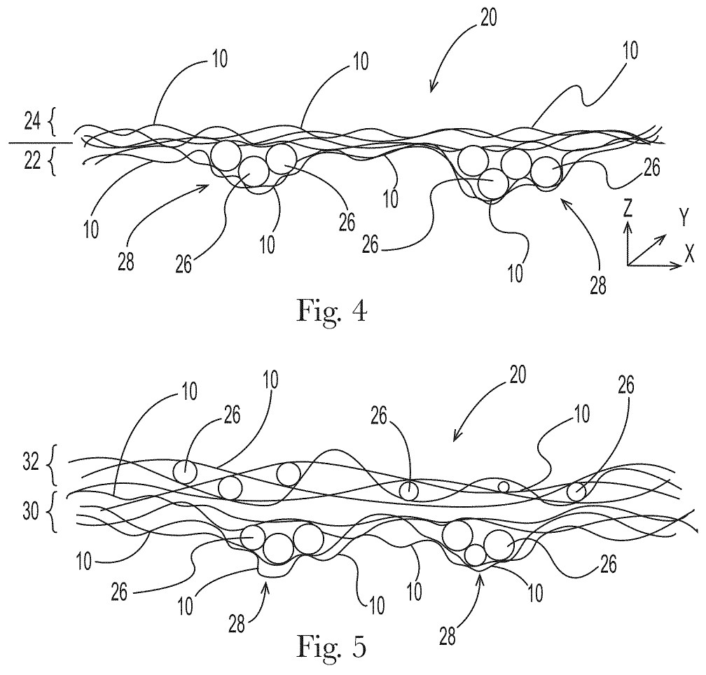

As shown in FIG. 4, another example of a dissolvable solid structure 20, for example a fibrous structure of the present invention comprises a first fibrous structure layer 22 comprising a plurality of fibrous elements, for example filaments 10, wherein the first fibrous structure layer 22 comprises one or more pockets 28 (also referred to as recesses, unfilled domes, or deflected zones), which may be in an irregular pattern or a non-random, repeating pattern. One or more of the pockets 28 may contain one or more particles 26. The dissolvable solid structure 20 in this example further comprises a second fibrous structure layer 24 that is associated with the first fibrous structure layer 22 such that the particles 26 are entrapped in the pockets 28. Like above, a similar dissolvable solid structure can be formed by depositing a plurality of particles in pockets of a first ply of fibrous structure comprising a plurality of fibrous elements and then associating a second ply of fibrous structure comprising a plurality of fibrous elements such that the particles are entrapped within the pockets of the first ply. In one example, the pockets may be separated from the fibrous structure to produce discrete pockets.

As shown in FIG. 5, another example of an dissolvable solid structure 20, for example a multi-ply fibrous structure of the present invention comprises a first ply 30 of a fibrous structure according to FIG. 4 above and a second ply 32 of fibrous structure associated with the first ply 30, wherein the second ply 32 comprises a plurality of fibrous elements, for example filaments 10, and a plurality of particles 26 dispersed, in this case randomly, in the x, y, and z axes, throughout the dissolvable solid structure 20.

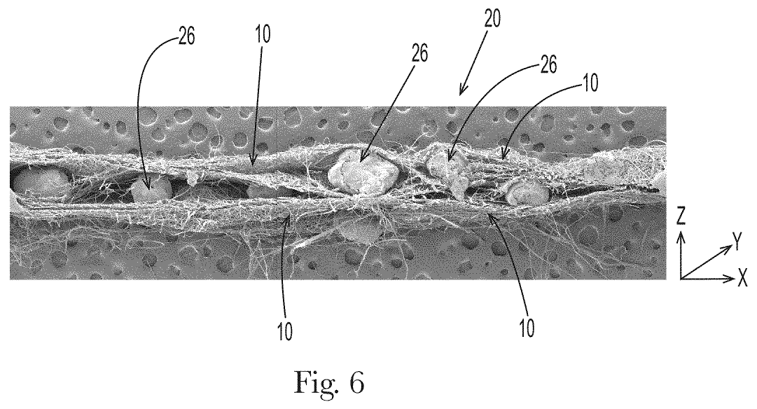

As shown in FIG. 6, another example of a dissolvable solid structure 20, for example a fibrous structure of the present invention comprises a plurality of fibrous elements, for example filaments 10, such as active agent-containing filaments, and a plurality of particles 26, for example active agent-containing particles, dispersed, in this case randomly, in the x, y, and z axes, throughout the fibrous structure of the dissolvable solid structure 20.

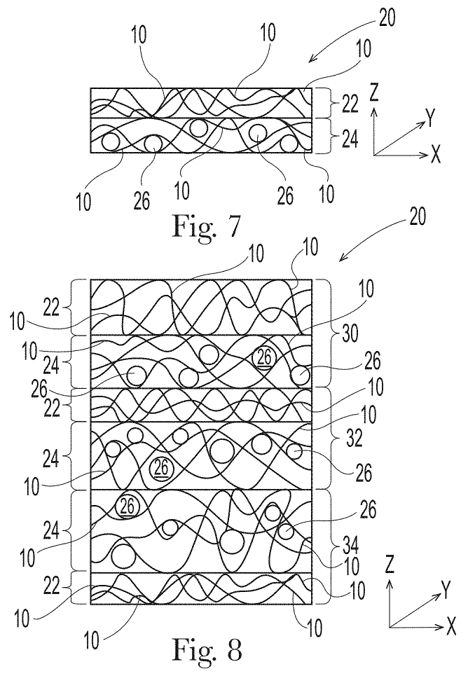

As shown in FIG. 7, another example of a dissolvable solid structure 20, for example a fibrous structure of the present invention comprises a first fibrous structure layer 22 comprising a plurality of fibrous elements, for example filaments 10, and a second fibrous structure layer 24 comprising a plurality of fibrous elements, for example filaments 10, for example active agent-containing filaments, and a plurality of particles 26, for example active agent-containing particles, dispersed, in this case randomly, in the x, y, and z axes, throughout the second fibrous structure layer 24. Alternatively, in another embodiment, the plurality of particles 26, for example active agent-containing particles, may be dispersed in an irregular pattern or a non-random, repeating pattern within the second fibrous structure layer 24. Like above, a similar dissolvable solid structure comprising two plies of fibrous structure comprising a first fibrous structure ply 22 comprising a plurality of fibrous elements, for example filaments 10, and a second fibrous structure ply 24 comprising a plurality of fibrous elements, for example filaments 10, for example active agent-containing filaments, and a plurality of particles 26, for example active agent-containing particles, dispersed, in this case randomly, in the x, y, and z axes, throughout the second fibrous structure ply 24. Alternatively, in another embodiment, the plurality of particles 26, for example active agent-containing particles, may be dispersed in an irregular pattern or a non-random, repeating pattern within the second fibrous structure ply 24.

FIG. 8 shows another example of a dissolvable solid structure 20, for example a multi-ply fibrous structure of the present invention comprising a first ply 30 of a fibrous structure as shown in FIG. 7 comprising a first fibrous structure layer 22 comprising a plurality of fibrous elements, for example filaments 10, and a second fibrous structure layer 24 comprising a plurality of fibrous elements, for example filaments 10, for example active agent-containing filaments, and a plurality of particles 26, for example active agent-containing particles, dispersed, in this case randomly, in the x, y, and z axes, throughout the second fibrous structure layer 24, a second ply 32 of a fibrous structure associated with the first ply 30, wherein the second ply 32 comprises a first fibrous structure layer 22 comprising a plurality of fibrous elements, for example filaments 10, and a second layer 24 comprising a plurality of fibrous elements, for example filaments 10, for example active agent-containing filaments, and a plurality of particles 26, for example active agent-containing particles, dispersed, in this case randomly, in the x, y, and z axes, throughout the second fibrous structure layer 24, and a third ply 34 of a fibrous structure associated with the second ply 32, wherein the third ply 34 comprises a first fibrous structure layer 22 comprising a plurality of fibrous elements, for example filaments 10, and a second fibrous structure layer 24 comprising a plurality of fibrous elements, for example filaments 10, for example active agent-containing filaments, and a plurality of particles 26, for example active agent-containing particles, dispersed, in this case randomly, in the x, y, and z axes, throughout the second fibrous structure layer 24.

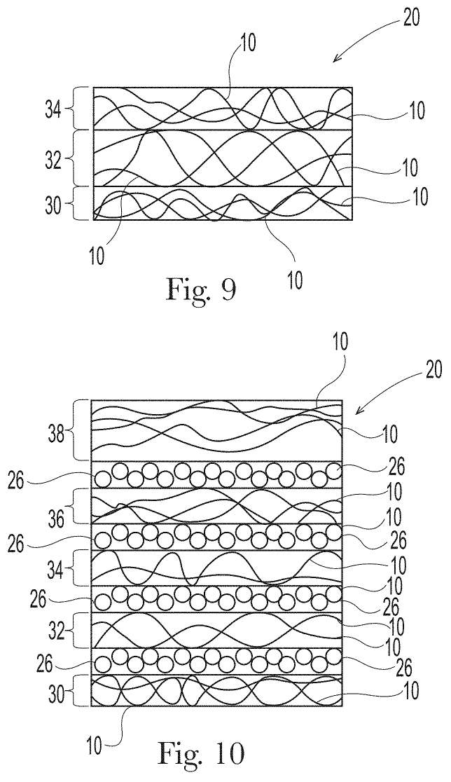

As shown in FIG. 9, another example of a dissolvable solid structure 20, for example a multi-ply fibrous structure of the present invention comprises a first ply 30 of a fibrous structure comprising a plurality of fibrous elements, for example filaments 10, a second ply 32 of a fibrous structure associated with the first ply 30, wherein the second ply 32 comprises a plurality of fibrous elements, for example filaments 10, and a third ply 34 of a fibrous structure associated with the second ply 32, wherein the third ply 34 comprises a plurality of fibrous elements, for example filaments 10. In one embodiment of FIG. 9, each ply's filaments 10 may comprise active agent-containing filaments.

FIG. 10 shows another example of a dissolvable solid structure 20 multi-ply fibrous structure 20 of the present invention comprising a first ply 30 of a fibrous structure comprising a plurality of fibrous elements, for example filaments 10, a second ply 32 of fibrous structure comprising a plurality of fibrous elements, for example filaments 10, a third ply 34 of a fibrous structure comprising a plurality of fibrous elements, for example filaments 10, a fourth ply 36 of fibrous structure comprising a plurality of fibrous elements, for example filaments 10, and a fifth ply 38 of a fibrous structure comprising a plurality of fibrous elements, for example filaments 10. In this example, the dissolvable solid structure 20 further comprises one or more particles or particle layers 26 positioned between at least two adjacent fibrous structure plies, for example plies 30 and 32 or plies 32 and 34 or plies 34 and 36 or plies 36 and 38. The plies 30, 32, 34, 36, and 38 are associated with one or more other plies to form a unitary structure and to minimize particles 26, if any are present within the dissolvable solid structure 20, from becoming disassociated from the dissolvable solid structure 20. In another embodiment, the one or more particles or particle layers 26 positioned between at least two adjacent fibrous structure plies are present in an irregular pattern, a non-random, repeating pattern, or only in select zones between the plies.

The term “molecular weight” or “Molecular weight” refers to the weight average molecular weight unless otherwise stated. Molecular weight is measured using industry standard method, gel permeation chromatography (“GPC”). As used herein, the articles including “a” and “an” when used in a claim, are understood to mean one or more of what is claimed or described.

As used herein, the terms “include,” “includes,” and “including,” are meant to be non-limiting.

The methods disclosed in the Test Methods Section of the present application should be used to determine the respective values of the parameters of Applicants' inventions.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### B. Dissolvable Solid Structure

Hair Care products in the form of a dissolvable solid structure present an attractive form to consumers. A typical use of these products includes a consumer holding the product in her hand, adding water to create a solution or dispersion and applying to the hair. In many cases, the products can take a long time to dissolve making it a less enjoyable experience for the consumer. Therefore, a need exists to have dissolvable solids that exhibit more rapid dissolution. Additionally, it is desirable to have a dissolvable solid structure that forms a lamellar structure upon addition of water to the dissolvable solid structure in the ratio of about 5:1.

In order to hydrate rapidly, the solid form conditioner can be created from fibrous structures with a relatively small fiber diameter, for instance less than 25 microns. However, the fibrous structures must have both strength and solubility/dispersibility in water. Typically, strength is achieved via the filament forming materials that can contain polymeric structurants with higher molecular weight exhibiting higher strength. However, high molecular weight polymers can be incompatible with additives, including active agents like conditioning agents such as high melting point fatty compounds and cationic surfactants which make them difficult for fiber spinning. Additionally, conditioning agents in the form of lamellar structures can cause defects in the fibers causing them to break.

It was surprisingly found that dissolvable solid structures that form a conditioning product in situ when exposed to water can be provided by combining conditioning agents, that can include high melting point fatty compounds and one or more cationic surfactants, with filament forming materials, which can include water-soluble polymeric structurants that are compatible with lamellar structures and have a sufficiently high molecular weight to create a strong fibrous form before exposure to water.

### Dissolvable Structures—Compositional

The Dissolvable Solid Structure can be a fibrous structure. The fibrous structure can contain a plurality of fibers and/or filaments. The fibers and/or filaments can contain one or more filament forming materials, additives including conditioning agents and optionally other additives, and one or more polar solvents.

### Filament-Forming Materials

To improve the fiber spinning of low viscosity material, such as molten fatty alcohols, fatty quaternary ammonium compounds, fatty acids, etc., a filament-forming material, such as a polymeric structurant can be added. The structurant can increase the shear and extensional viscosity of the fluid to enable fiber formation.

The filaments can be heterogeneous with globules containing dehydrated gel networks. When making heterogeneous filaments using the meltblowing technique, as described hereafter, the filaments are prone to forming nodules. If the nodules become too large, the filament can break into shorter filaments and/or fibers. The size and concentration of the nodule can be limited or eliminated by selecting certain polymeric structurants at certain levels to make the filament forming composition more polar, allowing better dissolution of the filament forming materials.

The structurant can be a high molecular weight species, for instance in the 10,000-5,000,000 g/mol range. A balance may be struck between concentration and molecular weight, such that when a lower molecular weight species is used, it may be incorporated at a higher level to function optimally. Likewise, when a higher molecular species is used, lower levels can be used to enable fiber spinning. To perform adequately, the structurants can be soluble in the continuous phase (e.g., water). The polymer solubility can enable the viscosity increase and subsequent fiber formation of the solution. Some nonlimiting examples of structurants that meet the above criteria are polyvinyl alcohol, polyethylene oxide, and polyvinylpyrrolidone. For example, the inventors have found that using PVOH 505 from Ashland Inc., having a molecular weight of about 40,000 g/mol is soluble in water and can enable fibers to be formed and collected onto a belt. It has been further found that polyethylene oxide with molecular weights of 100,000 and 2,000,000 also performed as suitable structurants but required different levels to be effective due to their molecular weight differences.

The Dissolvable Solid Structure, fibrous structure, filaments, and/or fibers can contain from about, 1% to about 75%, from about 5% to about 65%, from about 10% to about 60%, from about 15% to about 55%, from about 20% to about 50%, and/or from about 30% to about 45% by weight on a dry fibrous element basis and/or a dry fibrous structure basis and/or a dry filament basis of one or more filament forming materials.

The filament forming material can be a polymeric structurant. The filament-forming material can contain one or more substituted polymers such as an anionic, cationic, zwitterionic, and/or nonionic polymer.

The filament forming material can be a water-soluble polymeric structurant. As used herein, the term “water-soluble polymer” is broad enough to include both water-soluble and water-dispersible polymers, and is defined as a polymer with a solubility in water, measured at 25° C., of at least about 0.1 gram/liter (g/L). In some embodiments, the polymers have solubility in water, measured at 25° C., of from about 0.1 gram/liter (g/L). to about 500 grams/liter (g/L). (This indicates production of a macroscopically isotropic or transparent, colored or colorless solution). The polymers for making these Dissolvable Solid Structures may be of synthetic or natural origin and may be modified by means of chemical reactions. They may or may not be film-forming. These polymers should be physiologically acceptable, i.e., they should be compatible with the skin, mucous membranes, the hair and/or the scalp.

The one or more water-soluble polymers can be selected such that their weight average molecular weight is from about 10,000 g/mol to about 500,000 g/mol, alternatively from about 20,000 g/mol to about 500,000 g/mol, alternatively from about 30,000 g/mol to about 300,000 g/mol, alternatively from about 40,000 g/mol to about 200,000 g/mol. The one or more water-soluble polymers can be selected such that their weight average molecular weight is from about 25,000 g/mol to about 3,000,000 g/mol, alternatively from about 30,000 g/mol to about 2,500,000 g/mol, alternatively from about 40,000 g/mol to about 2,000,000 g/mol, alternatively from about 50,000 g/mol to 500,000 g/mol, and alternatively from about 65,000 g/mol to about 200,000 g/mol. The weight average molecular weight is computed by summing the average molecular weights of each polymer raw material multiplied by their respective relative weight percentages by weight of the total weight of polymers present within the Dissolvable Solid Structure.

When selecting water-soluble polymers, it can be important to strike a balance is between concentration and molecular weight, such that when a lower molecular weight species is used, it requires a higher level to result in optimal fiber spinning. Likewise, when a higher molecular species is used, lower levels can be used to achieve optimal fiber spinning. For instance, if the water-soluble polymer has a weight average molecular weight of from about 3,000,000 g/mol to about 5,000,000 g/mol it can be included at a level of from about 3 wt % to about 6 wt %, on a dry fibrous element basis and/or dry fibrous structure basis and/or a dry filament basis. Alternatively, a water-soluble polymer having a weight average molecular weight of from about 50,000 g/mol to about 100,000 g/mol can be included at a level of from about 30 wt % to about 50 wt %, on a dry fibrous element basis and/or dry fibrous structure basis and/or a dry filament basis.

The water-soluble polymer(s) can include, but are not limited to, synthetic polymers as described in U.S. Pat. No. 8,415,287 including polymers derived from acrylic monomers such as the ethylenically unsaturated carboxylic monomers and ethylenically unsaturated monomers as described in U.S. Pat. No. 5,582,786 and EP397410. The water-soluble polymer(s) which are suitable may also be selected from naturally sourced polymers including those of plant origin examples which are described in U.S. Pat. No. 8,415,287. Modified natural polymers are also useful as water-soluble polymer(s) and are included in U.S. Pat. No. 8,415,287.

The water-soluble polymers can include a hydroxyl polymer such as a polyvinyl alcohol (“PVOH”), a partially hydrolyzed polyvinyl acetate and/or a polysaccharide, polyacrylates, polymethacrylates, copolymers of acrylic acid and methyl acrylate, polyvinylmethylether, polyvinylformamide, polyacrylamide, polyalkylene oxides, starch and starch derivatives, pullulan, gelatin, rayon, modified celluloses including hydroxypropylmethylcelluloses, methycelluloses, hydroxypropyl cellulose, hydroxyethyl cellulose, and carboxymethycelluloses, salts and combinations thereof. In another embodiment, water-soluble polymers can include polyvinyl alcohols, and hydroxypropylmethylcelluloses. Suitable polyvinyl alcohols include those available from Celanese Corporation (Dallas, Tex.) under the CELVOL® trade name. Suitable hydroxypropylmethylcelluloses include those available from the Dow Chemical Company (Midland, Mich.) under the METHOCEL® trade name.

In one example, the water-soluble polymer can be a vinyl acetate-vinyl alcohol copolymer. As used herein. “vinyl acetate-vinyl alcohol copolymer” (and “copolymer” when used in reference thereto) refers to a polymer of the following structure (I):

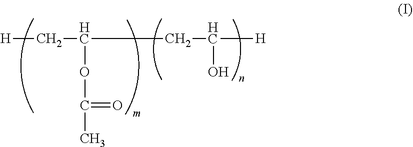

In structure (I), m and n are integers such that the polymeric structurant has the degree of polymerization and percent alcohol characteristics described herein. For purposes of clarity, this use of the term “copolymer” is intended to convey that the partially hydrolyzed polyvinyl acetate of the present invention comprises vinyl alcohol and vinyl acetate units. As discussed below, the polymeric structurant is routinely prepared by polymerizing vinyl acetate monomer followed by hydrolysis of some of the acetate groups to alcohol groups, as opposed to polymerization of vinyl acetate and vinyl alcohol monomer units (due in-part to the instability of vinyl alcohol).

In certain examples, the filament-forming composition can contain non-starch structurants (e.g. polyvinyl alcohol, polyethylene oxide) and any single starch-based material or combination of in such an amount as to reduce the overall level of water-soluble polymers needed, so long as it helps provide the Dissolvable Solid Structure with the requisite structure and physical/chemical characteristics as described herein. In such instances, the combined weight percentage of the non-starch structurant(s) and starch-based material can range from about 10% to about 75%, alternatively from about 15% to about 40%, and alternatively from about 20% to about 30%, by weight on a dry fibrous element basis and/or a dry fibrous structure basis and/or a dry filament basis. The weight ratio of the non-starch polymer(s) to the starch-based material can generally range from about 1:10 to about 10:1, alternatively from about 1:8 to about 8:1, alternatively from about 1:7 to about 7:1, and alternatively from about 6:1 to about 1:6.

Typical sources for starch-based materials can include cereals, tubers, roots, legumes and fruits. Native sources can include corn, pea, potato, banana, barley, wheat, rice, sago, amaranth, tapioca, arrowroot, canna, sorghum, and waxy or high amylase varieties thereof. The starch-based materials may also include native starches that are modified using any modification known in the art, including those described in U.S. Pat. No. 8,415,287.

In some examples, the filament forming material can be soluble in an oily mixture to enable viscosity build for fiber spinning. In certain examples, the filament forming materials can be water-soluble and oil soluble. Suitable structurants include, but are not limited to, polyvinylpyrrolidone, polydimethylacrylamides, and combinations thereof. Additional suitable polymers include copolymers of polyvinylpyrrolidone, such as Ganex® or PVP/VA (weight average molecular weight of about 50,000 g/mol) copolymers from Ashland Inc., also performed as suitable structurants but a higher level was utilized to be effective due to their lower weight average molecular weight. In addition, copolymers of polydimethylacrylamide also function as a suitable structurant. Hydroxyl propyl cellulose can also function as a suitable structurant. Additional oil soluble structurants include those described in U.S. Ser. No. 62/506,777.

In yet another example, the filament-forming material is a polar solvent-soluble material. Alternatively, the filament forming material can contain polyethylene oxide (PEO) polymers.

Additional examples of filament-forming materials can be found in US2012/0058166.

### Additives

The fibers and/or filaments can one or more additives including conditioning agents and optionally other additives.

Non-limiting examples of conditioning agents can include high melting point fatty compounds, cationic surfactants, and other suitable conditioning agents including silicones, such as functionalized silicones, a high charge density cationic synthetic polymer, such as polydiallyldimethylammonium chloride (polyDADMAC), a cationic guar polymer, and combinations thereof. The filament-forming material can contain high melting point fatty compounds and cationic surfactants.

Other additives can include dispersing agents, plasticizers, optional ingredients, and combinations thereof.

Examples of other suitable conditioning agents are discussed in US 2011/0135588, US 2008/0019935, US 2008/0242584, US 2006/0217288, U.S. Pat. Nos. 9,662,291, and 8,435,501.

### Cationic Surfactant

The dissolvable solid structure, fibrous structure, filaments, and/or fibers can contain from about 1% to about 25%, about 2% to about 20%, about 3% to about 15%, about 4% to about 13%, and about 5% to about 10% by weight on a dry fibrous element basis and/or a dry fibrous structure basis and/or a dry filament basis of one or more cationic surfactants.

Cationic surfactant useful herein can be one cationic surfactant or a mixture of two or more cationic surfactants. The cationic surfactant can be selected from the group consisting of, but not limited to: a mono-long alkyl quaternized ammonium salt; a combination of a mono-long alkyl quaternized ammonium salt and a di-long alkyl quaternized ammonium salt; a mono-long alkyl amine; a combination of a mono-long alkyl amine and a di-long alkyl quaternized ammonium salt; and a combination of a mono-long alkyl amine and a mono-long alkyl quaternized ammonium salt, a tertiary amine and combinations thereof.

### Mono-Long Alkyl Amine

Mono-long alkyl amine useful herein are those having one long alkyl chain of from 12 to 30 carbon atoms, alternatively from 16 to 24 carbon atoms, alternatively from 18 to 22 alkyl group. Mono-long alkyl amines useful herein also include mono-long alkyl amidoamines. Primary, secondary, and tertiary fatty amines are useful.

Suitable for use in the dissolvable solid structure are tertiary amido amines having an alkyl group of from about 12 to about 22 carbons. Exemplary tertiary amido amines include: stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide. Useful amines in the present invention are disclosed in U.S. Pat. No. 4,275,055, Nachtigal, et al.

These amines can be used in combination with acids such as l-glutamic acid, lactic acid, hydrochloric acid, malic acid, succinic acid, acetic acid, fumaric acid, tartaric acid, citric acid, l-glutamic hydrochloride, maleic acid, and mixtures thereof; alternatively l-glutamic acid, lactic acid, citric acid, at a molar ratio of the amine to the acid of from about 1:0.3 to about 1:2, alternatively from about 1:0.4 to about 1:1.

### Mono-Long Alkyl Quaternized Ammonium Salt

The mono-long alkyl quaternized ammonium salts useful herein are those having one long alkyl chain which has from 12 to 30 carbon atoms, alternatively from 16 to 24 carbon atoms, alternatively a C18-22 alkyl group. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Mono-long alkyl quaternized ammonium salts useful herein are those having the formula

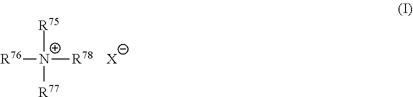

wherein one of R⁷⁵, R⁷⁶, R⁷⁷ and R⁷⁸ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. One of R⁷⁵, R⁷⁶, R⁷⁷ and R⁷⁸ can be selected from an alkyl group of from 12 to 30 carbon atoms, alternatively from 16 to 24 carbon atoms, alternatively from 18 to 22 carbon atoms, alternatively 22 carbon atoms; the remainder of R⁷⁵, R⁷⁶, R⁷⁷ and R⁷⁸ can be independently selected from CH₃, C₂H₅, C₂H₄OH, and mixtures thereof; and X can be selected from the group consisting of Cl, Br, CH₃OSO₃, C₂H₅OSO₃, and mixtures thereof.

Nonlimiting examples of such mono-long alkyl quaternized ammonium salt cationic surfactants include: behenyl trimethyl ammonium salt; stearyl trimethyl ammonium salt; cetyl trimethyl ammonium salt; and hydrogenated tallow alkyl trimethyl ammonium salt.

### Di-Long Alkyl Quaternized Ammonium Salts

When used, di-long alkyl quaternized ammonium salts can be combined with a mono-long alkyl quaternized ammonium salt and/or mono-long alkyl amine salt, at the weight ratio of from 1:1 to 1:5, alternatively from 1:1.2 to 1:5, alternatively from 1:1.5 to 1:4, in view of stability in rheology and conditioning benefits.

Di-long alkyl quaternized ammonium salts useful herein are those having two long alkyl chains of from 12 to 30 carbon atoms, alternatively from 16 to 24 carbon atoms, alternatively from 18 to 22 carbon atoms. Such di-long alkyl quaternized ammonium salts useful herein are those having the formula (I):

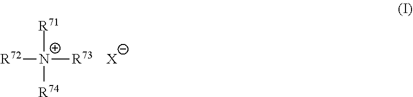

wherein two of R⁷¹, R⁷², R⁷³ and R⁷⁴ are selected from an aliphatic group of from 12 to 30 carbon atoms, alternatively from 16 to 24 carbon atoms, alternatively from 18 to 22 carbon atoms or an aromatic, alkoxy, polyoxyalkylene, alkylamido, hydroxyalkyl, aryl or alkylaryl group having up to about 30 carbon atoms; the remainder of R⁷¹, R⁷², R⁷³ and R⁷⁴ are independently selected from an aliphatic group of from 1 to about 8 carbon atoms, alternatively from 1 to 3 carbon atoms or an aromatic, alkoxy, polyoxyalkylene, alkylamido, hydroxyalkyl, aryl or alkylaryl group having up to about 8 carbon atoms; and X⁻ is a salt-forming anion selected from the group consisting of halides such as chloride and bromide, C1-C4 alkyl sulfate such as methosulfate and ethosulfate, and mixtures thereof. The aliphatic groups can contain, in addition to carbon and hydrogen atoms, ether linkages, and other groups such as amino groups. The longer chain aliphatic groups, e.g., those of about 16 carbons, or higher, can be saturated or unsaturated. Two of R⁷¹, R⁷², R⁷³ and R⁷⁴ can be selected from an alkyl group of from 12 to 30 carbon atoms, alternatively from 16 to 24 carbon atoms, alternatively from 18 to 22 carbon atoms; and the remainder of R⁷¹, R⁷², R⁷³ and R⁷⁴ are independently selected from CH₃, C₂H₅, C₂H₄OH, CH₂C₆H₅, and mixtures thereof.

Suitable di-long alkyl cationic surfactants include, for example, dialkyl (14-18) dimethyl ammonium chloride including dicetyldimonium chloride, ditallow alkyl dimethyl ammonium chloride, dihydrogenated tallow alkyl dimethyl ammonium chloride, distearyl dimethyl ammonium chloride, and dicetyl dimethyl ammonium chloride.

### High Melting Point Fatty Compound

The dissolvable solid structure, fibrous structure, filaments, and/or fibers can contain from about 3% to about 45%, about 5% to about 40%, about 7% to about 37%, about 10% to about 34%, about 12% to about 30%, and/or about 15% to about 27% by weight on a dry fibrous element basis and/or a dry fibrous structure basis and/or a dry filament basis of one or more high melting point fatty compounds.

The fatty compound can be selected from the group consisting of, but not limited to, fatty amphiphiles, fatty alcohol, fatty acid, fatty amide, fatty ester and combinations thereof.

The high melting point fatty compound useful herein have a melting point of 25° C. or higher, alternatively 40° C. or higher, alternatively 45° C. or higher, alternatively 50° C. or higher, in view of stability of the emulsion especially the gel matrix. Such melting point is up to about 90° C., alternatively up to about 80° C., alternatively up to about 70° C., alternatively up to about 65° C., in view of easier manufacturing and easier emulsification. The high melting point fatty compound can be used as a single compound or as a blend or mixture of at least two high melting point fatty compounds. When used as such blend or mixture, the above melting point means the melting point of the blend or mixture.

The high melting point fatty compound useful herein is selected from the group consisting of fatty alcohols, fatty acids, fatty alcohol derivatives, fatty acid derivatives, fatty amides, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than the above. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

Among a variety of high melting point fatty compounds, fatty alcohols can be used in the composition described herein. The fatty alcohols useful herein are those having from about 14 to about 30 carbon atoms, alternatively from about 16 to about 22 carbon atoms. These fatty alcohols are saturated and can be straight or branched chain alcohols.

Suitable fatty alcohols include, but are not limited to, cetyl alcohol (having a melting point of about 56° C.), stearyl alcohol (having a melting point of about 58-59° C.), behenyl alcohol (having a melting point of about 71° C.), and mixtures thereof. These compounds are known to have the above melting point. However, they often have lower melting points when supplied, since such supplied products are often mixtures of fatty alcohols having alkyl chain length distribution in which the main alkyl chain is cetyl, stearyl or behenyl group.

Generally, in the mixture, the weight ratio of cetyl alcohol to stearyl alcohol is from about 1:9 to 9:1, alternatively from about 1:4 to about 4:1, alternatively from about 1:2.3 to about 1.5:1.

When using higher level of total cationic surfactant and high melting point fatty compounds, the mixture has the weight ratio of cetyl alcohol to stearyl alcohol of from about 1:1 to about 4:1, alternatively from about 1:1 to about 2:1, alternatively from about 1.2:1 to about 2:1, in view of maintaining acceptable consumer usage. It may also provide more conditioning on damaged part of the hair.

### Dispersing Agents

The dissolvable solid structure, fibrous structure, filaments, and/or fibers can contain from about 1% to about 30%, about 5% to about 15%, and/or about 5% to about 10%, by weight on a dry fibrous element basis and/or a dry fibrous structure basis and/or a dry filament basis of one or more dispersing agents.

When preparing dissolvable solid structure, it has been found that the addition of a dispersing agent greatly increases the wetting, hydration, and dispersion of the conditioner materials. A surfactant from the nonionic class of alkyl glucamides can improve the wetting and hydration when added to the solid conditioner formula. The alkyl glucamide surfactant contains a hydrophobic tail of about 8-18 carbons and a nonionic head group of glucamide. For glucamide, the presence of the amide and hydroxyl groups may provide sufficient polarity that balances the hydrophobic carbon tail in such a way to permit the surfactant's solubility in the conditioner oils and also imparts a rapid dispersion of the conditioner ingredients upon exposure to water. Other similar dispersing agents include, but are not limited to, reverse alkyl glucamides, cocoamiodpropyl betaines, alkyl glucoside, Triethanol amine, cocamide MEAs and mixtures thereof.

### Plasticizer

The Dissolvable Solid Structure, fibrous structure, filaments, and/or fibers can contain from about 0.5% to about 25%, about 3% to about 20%, from about 5% to about 15% by weight on a dry fibrous element basis and/or a dry fibrous structure basis and/or a dry filament basis of one or more plasticizers.

When present, non-limiting examples of suitable plasticizing agents can include polyols, copolyols, polycarboxylic acids, polyesters and dimethicone copolyols.

Examples of useful polyols can include, but are not limited to, glycerin, diglycerin, propylene glycol, ethylene glycol, butylene glycol, pentylene glycol, cyclohexane dimethanol, hexane diol, polyethylene glycol (200-600), sugar alcohols such as sorbitol, mannitol, lactitol, isosorbide, glucamine, N-methylglucamine and other mono- and polyhydric low molecular weight alcohols (e.g., C₂-C₈ alcohols); mono di- and oligo-saccharides such as fructose, glucose, sucrose, maltose, lactose, and high fructose corn syrup solids and ascorbic acid.

Examples of polycarboxylic acids include, but are not limited to citric acid, maleic acid, succinic acid, polyacrylic acid, and polymaleic acid.

Examples of suitable polyesters include, but are not limited to, glycerol triacetate, acetylated-monoglyceride, diethyl phthalate, triethyl citrate, tributyl citrate, acetyl triethyl citrate, acetyl tributyl citrate.

Examples of suitable dimethicone copolyols include, but are not limited to, PEG-12 dimethicone, PEG/PPG-18/18 dimethicone, and PPG-12 dimethicone.

Other suitable plasticizers include, but are not limited to, alkyl and allyl phthalates; napthalates; lactates (e.g., sodium, ammonium and potassium salts); sorbeth-30; urea; lactic acid; sodium pyrrolidone carboxylic acid (PCA); sodium hyraluronate or hyaluronic acid; soluble collagen; modified protein; monosodium L-glutamate; alpha & beta hydroxyl acids such as glycolic acid, lactic acid, citric acid, maleic acid and salicylic acid; glyceryl polymethacrylate; polymeric plasticizers such as polyquaterniums; proteins and amino acids such as glutamic acid, aspartic acid, and lysine; hydrogen starch hydrolysates; other low molecular weight esters (e.g., esters of C₂-C₁₀ alcohols and acids); and any other water-soluble plasticizer known to one skilled in the art of the foods and plastics industries; and mixtures thereof.

EP 0283165 BI discloses suitable plasticizers, including glycerol derivatives such as propoxylated glycerol.

### Additional Optional Ingredients

The Dissolvable Solid Structure, fibrous structure, filaments, and/or fibers can contain other optional ingredients that are known for use or otherwise useful in compositions, provided that such optional materials are compatible with the selected essential materials described herein, or do not otherwise unduly impair product performance. The optional ingredients can be incorporated into the filament-forming composition or they can be applied after the fibers and/or filaments and/or structure has been formed.

Such optional ingredients are most typically those materials approved for use in cosmetics and that are described in reference books such as the CTFA Cosmetic Ingredient Handbook, Second Edition, The Cosmetic, Toiletries, and Fragrance Association, Inc. 1992.

Emulsifiers suitable as an optional ingredient herein include mono- and di-glycerides, fatty alcohols, polyglycerol esters, propylene glycol esters, sorbitan esters and other emulsifiers known or otherwise commonly used to stabilized air interfaces, as for example those used during preparation of aerated foodstuffs such as cakes and other baked goods and confectionary products, or the stabilization of cosmetics such as hair mousses.

Further non-limiting examples of such optional ingredients include preservatives, perfumes or fragrances, coloring agents or dyes, conditioning agents, hair bleaching agents, thickeners, moisturizers, emollients, pharmaceutical actives, vitamins or nutrients, sunscreens, deodorants, sensates, plant extracts, nutrients, astringents, cosmetic particles, absorbent particles, adhesive particles, hair fixatives, fibers, reactive agents, skin lightening agents, skin tanning agents, anti-dandruff agents, perfumes, exfoliating agents, acids, bases, humectants, enzymes, suspending agents, pH modifiers, hair colorants, hair perming agents, pigment particles, anti-acne agents, anti-microbial agents, sunscreens, tanning agents, exfoliation particles, hair growth or restorer agents, insect repellents, shaving lotion agents, co-solvents or other additional solvents, and similar other materials. Further non-limiting examples of optional ingredients include encapsulated perfumes, such as by 1-cyclodetrins, polymer microcapsules, starch encapsulated accords and combinations thereof.

### Volatile Solvent

The Dissolvable Solid Structure, fibrous structure, filaments, and/or fibers can contain one or more volatile solvents. Non-limiting examples of a volatile solvent can be selected from the group consisting of water, acetic acid, acetone methanol, ethanol, propanol, isopropyl alcohol, butanol, methylethyl ketones, and combinations thereof.

The dissolvable solid structure, fibrous structure, filaments, and/or fibers can contain 10% or less, 7% or less, 5% or less, and/or 4% by weight on a dry fibrous element basis and/or a dry fibrous structure basis and/or a dry filament basis of volatile solvent. The dissolvable solid structure, fibrous structure, filaments, and/or fibers can contain from about 0.1% to about 10%, from about 0.5% to about 6%, from about 1% to about 5%, and/or from about 1% to about 4% by weight on a dry fibrous element basis and/or a dry fibrous structure basis and/or dry filament basis of volatile solvent.

### Dissolvable Solid Structure

Non-limiting examples of product type embodiments for use by the Structure include hand cleansing Structures, hair shampoo or other hair treatment Structures, body cleansing Structures, shaving preparation Structures, personal care Structures containing pharmaceutical or other skin care active, moisturizing Structures, sunscreen Structures, chronic skin benefit agent Structures (e.g., vitamin-containing Structures, alpha-hydroxy acid-containing Structures, etc.), deodorizing Structures, fragrance-containing Structures, and so forth.

For fibrous Structures, the Structure comprises a significant number of dissolvable fibers with an average diameter less than about 150 micron, alternatively less than about 100 micron, alternatively less than about 10 micron, and alternatively less than about 1 micron with a relative standard deviation of less than 100%, alternatively less than 80%, alternatively less than 60%, alternatively less than 50%, such as in the range of 10% to 50%, for example. As set forth herein, the significant number means at least 10% of all the dissolvable fibers, alternatively at least 25% of all the dissolvable fibers, alternatively at least 50% of all the dissolvable fibers, alternatively at least 75% of all the dissolvable fibers. The significant number may be at least 99% of all the dissolvable fibers. Alternatively, from about 50% to about 100% of all the dissolvable fibers may have an average diameter less than about 10 micron. The dissolvable fibers produced by the method of the present disclosure have a significant number of dissolvable fibers with an average diameter less than about 1 micron, or sub-micron fibers. In an embodiment, Dissolvable Solid Structure may have from about 25% to about 100% of all the dissolvable fibers with an average diameter less than about 1 micron, alternatively from about 35% to about 100% of all the dissolvable fibers with an average diameter less than about 1 micron, alternatively from about 50% to about 100% of all the dissolvable fibers with an average diameter less than about 1 micron, and alternatively from about 75% to about 100% of all the dissolvable fibers with an average diameter less than about 1 micron.

The percent porosity of the dissolvable solid Structure is at least about 25%, alternatively at embodiment at least about 50%, alternatively at least about 60%, alternatively at least about 70% and alternatively at least about 80%. The porosity of the dissolvable solid Structure is not more than about 99%, alternatively not more than about 98%, alternatively not more than about 95%, and alternatively not more than about 90%. Porosity of a Structure is determined according to the procedure set forth in the definition of “porosity” below.

As used herein, “porosity” and “percent porosity” are used interchangeably and each refers to a measure of void volume of the Dissolvable Solid Structure and is calculated as

$$( 1 - \frac{Basis Weight of Dissolvable Solid Structure}{\begin{matrix}Thickness of Dissolvable Solid Structure \times \\ ρ of the bulk , dried material\end{matrix}} ) \times 100 \%$$

with the units adjusted so they cancel and multiplied by 100% to provide percent porosity.

A range of effective sizes of pores can be accommodated. The pore size distribution through the Structure cross-section may be symmetric or asymmetric.

The Structure can be flexible and have a distance to maximum force value of from about 6 mm to about 30 mm. The distance to maximum force value from about 7 mm to about 25 mm, alternatively from about 8 mm to about 20 mm, and alternatively from about 9 mm to about 15 mm.

The Structure can be characterized in one aspect by its Specific Surface Area. The Structure can have a Specific Surface Area of from about 0.03 m²/g to about 0.25 m²/g, alternatively from about 0.035 m²/g to about 0.22 m²/g, alternatively from about 0.04 m²/g to about 0.19 m²/g, and alternatively from about 0.045 m²/g to about 0.16 m²/g.

The Structure can be a flat, flexible Structure in the form of a pad, a strip, or tape and having a thickness of from about 0.5 mm to about 10 mm, alternatively from about 1 mm to about 9 mm, alternatively from about 2 mm to about 8 mm, and alternatively from about 3 mm to about 7 mm as measured by the below methodology. The Structure can be a sheet having a thickness from about 5 mm to about 6.5 mm. Alternatively two or more sheets are combined to form a Structure with a thickness of about 5 mm to about 10 mm.

The Structure can have a basis weight of from about 200 grams/m² to about 2,000 grams/m², alternatively from about 400 g/m² to about 1,200 g/m², alternatively from about 600 g/m² to about 2,000 g/m², and alternatively from about 700 g/m² to about 1,500 g/m².

The Structure can have a dry density of from about 0.08 g/cm³ to about 0.40 g/cm³, alternatively from about 0.08 g/cm³ to about 0.38 g/cm³, alternatively from about 0.10 g/cm³ to about 0.25 g/cm³, and alternatively from about 0.12 g/cm³ to about 0.20 g/cm³.

### Methods of Manufacture—Fibrous Structures

The fibrous elements of the present invention may be made by any suitable process.

The filament-forming composition can be spun into one or more fibrous elements and/or particles by any suitable spinning process, such as meltblowing, spunbonding, electro-spinning, and/or rotary spinning. In one example, the filament-forming composition is spun into a plurality of fibrous elements by meltblowing. For example, the filament-forming composition may be pumped from a tank to a meltblown spinnerette. Upon exiting one or more of the filament-forming holes in the spinnerette, the filament-forming composition is attenuated with air to create one or more fibrous elements and/or particles. The fibrous elements and/or particles may then be dried to remove any remaining solvent used for spinning, such as the water.

The fibrous elements and/or particles of the present invention may be collected on a belt, such as a patterned belt to form a fibrous structure comprising the fibrous elements and/or particles.

A non-limiting example of a suitable process for making the fibrous elements is described below.

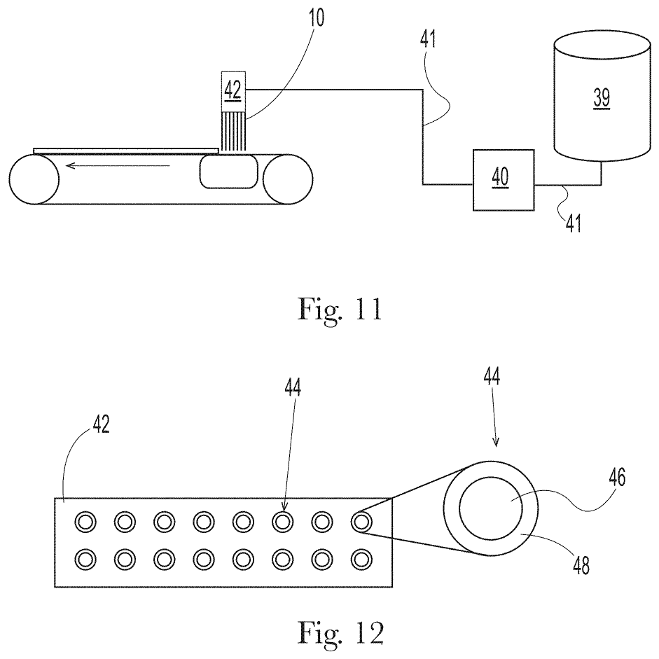

In one example, as shown in FIGS. 11 and 12, a method for making a fibrous element 10 can comprise the steps of:

a. providing a filament-forming composition as described herein; and

b. spinning the filament-forming composition, such as via a spinning die 42, into one or more fibrous elements 10.

As shown in FIGS. 11 and 12, the fibrous elements of the present invention may be made as follows. Fibrous elements 10 may be formed by means of a small-scale apparatus, a schematic representation of which is shown in FIGS. 11 and 12. A pressurized tank 39, suitable for batch operation is filled with a suitable filament-forming composition. A pump 40 such as a Zenith®, type PEP II, having a capacity of 5.0 cubic centimeters per revolution (cc/rev), manufactured by Parker Hannifin Corporation, Zenith Pumps division, of Sanford, N.C., USA may be used to facilitate transport of the filament-forming composition via pipes 41 to a spinning die 42. The flow of the filament-forming composition from the pressurized tank 39 to the spinning die 42 may be controlled by adjusting the number of revolutions per minute (rpm) of the pump 40. Pipes 41 are used to connect the pressurized tank 39, the pump 40, and the spinning die 42.

As shown in FIG. 12, the spinning die 42 may comprise a plurality of fibrous element-forming holes 44 that include a melt capillary 46 encircled by a concentric attenuation fluid hole 48 through which a fluid, such as attenuation air, passes to facilitate attenuation of the filament-forming composition into a fibrous element 10 as it exits the fibrous element-forming hole 44.

The spinning die 42 shown in FIG. 12 has several rows of circular extrusion nozzles (fibrous element-forming holes 44) spaced from one another at a pitch of about 1.524 millimeters (about 0.060 inches). The nozzles have individual inner diameters of about 0.305 millimeters (about 0.012 inches) and individual outside diameters of about 0.813 millimeters (about 0.032 inches).

Attenuation air can be provided by heating compressed air from a source by an electrical-resistance heater, for example, a heater manufactured by Chromalox, Division of Emerson Electric, of Pittsburgh, Pa., USA. An appropriate quantity of steam was added to saturate or nearly saturate the heated air at the conditions in the electrically heated, thermostatically controlled delivery pipe. Condensate is removed in an electrically heated, thermostatically controlled, separator.

The embryonic fibrous elements are dried by a drying air stream having a temperature from about 149° C. (about 300° F.) to about 315° C. (about 600° F.) by an electrical resistance heater (not shown) supplied through drying nozzles and discharged at an angle of about 90° relative to the general orientation of the embryonic fibrous elements being extruded. The dried embryonic fibrous elements are collected on a collection device, such as, for example, a movable foraminous belt or patterned collection belt. The addition of a vacuum source directly under the formation zone may be used to aid collection of the fibers.

In one example, during the spinning step, most of the volatile solvent, such as water, present in the filament-forming composition is removed, such as by drying, as the fibrous element 10 is formed. In one example, greater than 30% and/or greater than 40% and/or greater than 60%, and/or greater than 70%, and/or greater than 75%, and/or greater than 80%, and/or greater than 90%, and/or greater than 95%, of the weight of the filament-forming composition's volatile solvent, is removed during the spinning step, such as by drying the fibrous element being produced.

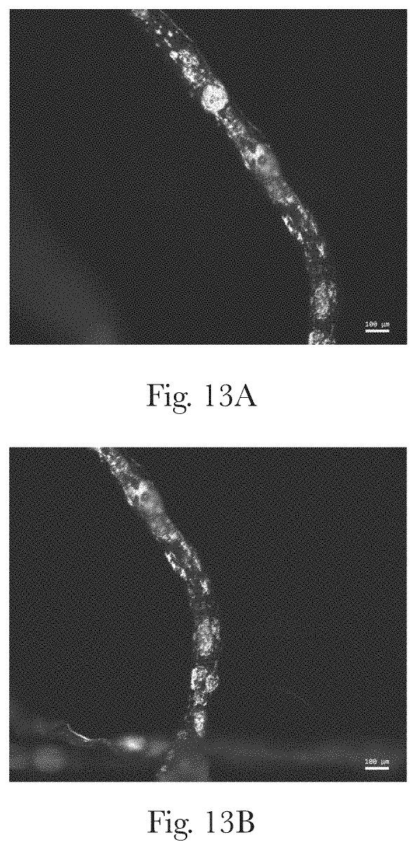

FIGS. 13A and 13B are light microscopy images of filaments using crossed-polars. The filaments in FIGS. 13A and 13B appear heterogeneous by microscopic inspection as determined by the Microscopic Inspection Method described hereafter. As can be seen in the images, the filaments have internal globules which show up with bright contrast relative to the surrounding filament at specific orientations of stage rotation relative to the crossed-polars. The globules contain a dehydrated gel network, which comprises the high melting point fatty compound(s) and cationic surfactant(s), and is a different composition, crystallinity and orientation than the remainder of the filament.

It can be desirable for the width of the globules to be less than or equal to the diameter of the filament. The globules can have a width of less than 25 microns, less than 20 microns, less than 15 microns, less than 10 microns, and/or less than 5 microns. The globules can have a width of from about 1 micron to about 30 microns, from about 3 microns to about 20 microns, from about 5 microns to about 17 microns, from about 7 microns to about 13 microns, and/or from about 8 microns to about 12 microns.

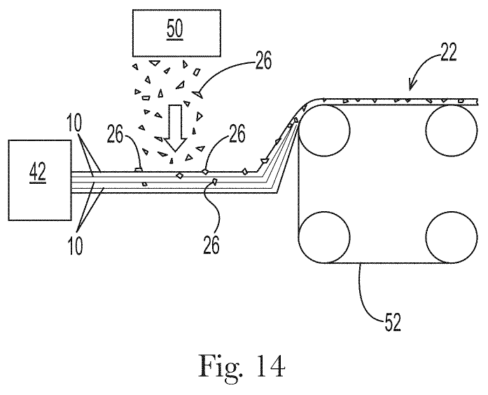

As shown in FIG. 14, a fibrous structure, for example a fibrous structure layer or ply 22 of the present invention may be made by spinning a filament-forming composition from a spinning die 42, as described in FIGS. 11 and 12, to form a plurality of fibrous elements, such as filaments 10, and then optionally, associating one or more particles 26 provided by a particle source 50, for example a sifter or an airlaid forming head. The particles 26 may be dispersed within the fibrous elements, for example filaments 10. The mixture of particles 26 and fibrous elements, for example filaments 10 may be collected on a collection belt 52, such as a patterned collection belt that imparts a texture, such as a three-dimensional texture to at least one surface of the fibrous structure layer or ply 22.

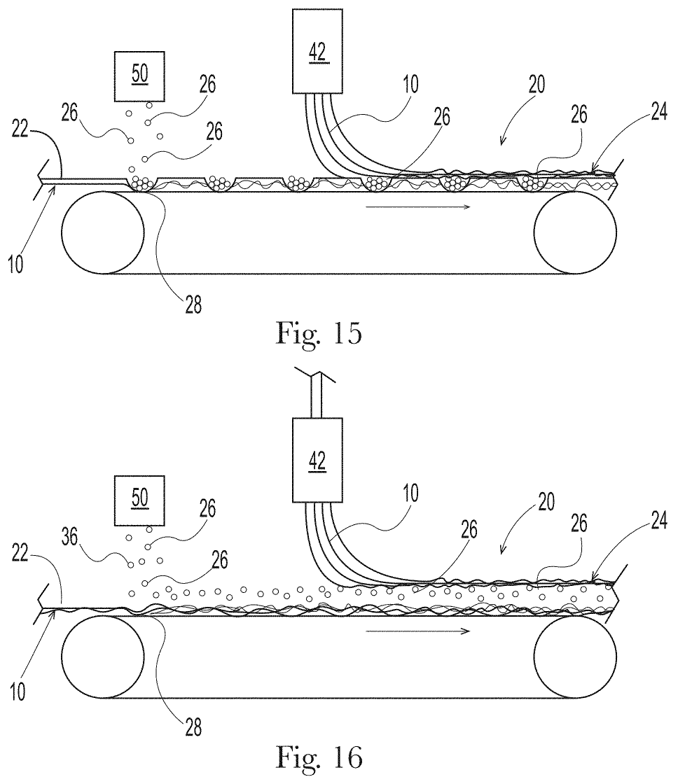

FIG. 15 illustrates an example of a method for making a dissolvable solid structure 20 according to FIG. 4. The method comprises the steps of forming a first fibrous structure layer 22 of a plurality of fibrous elements, for example filaments 10 such that pockets 28 are formed in a surface of the first fibrous structure layer 22. One or more particles 26 are deposited into the pockets 28 from a particle source 50. A second fibrous structure layer 24 comprising a plurality of fibrous elements, for example filaments 10 produced from a spinning die 42 are then formed on the surface of the first fibrous structure layer 22 such that the particles 26 are entrapped in the pockets 28.

FIG. 16 illustrates yet another example of a method for making a dissolvable solid structure 20 according to FIG. 3. The method comprises the steps of forming a first fibrous structure layer 22 of a plurality of fibrous elements, for example filaments 10. One or more particles 26 are deposited onto a surface of the first fibrous structure layer 22 from a particle source 50. A second fibrous structure layer 24 comprising a plurality of fibrous elements, for example filaments 10 produced from a spinning die 42 are then formed on top of the particles 26 such that the particles 26 are positioned between the first fibrous structure layer 22 and the second fibrous structure layer 24.

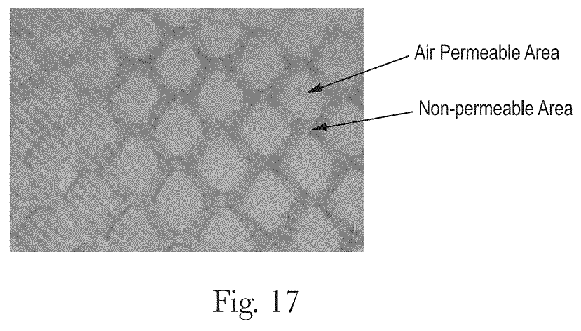

The dry embryonic fibrous elements, for example filaments may be collected on a molding member. The construction of the molding member may provide areas that are air-permeable due to the inherent construction. The filaments that are used to construct the molding member will be non-permeable while the void areas between the filaments will be permeable. Additionally, a pattern may be applied to the molding member to provide additional non-permeable areas which may be continuous, discontinuous, or semi-continuous in nature. A vacuum used at the point of lay down is used to help deflect fibers into the presented pattern. An example of one of these molding members is shown in FIG. 17.

In addition to the techniques described herein in forming regions within the fibrous structures having different properties (e.g., average densities), other techniques can also be applied to provide suitable results. One such example includes embossing techniques to form such regions. Suitable embossing techniques are described in U.S. Patent Application Publication Nos. 2010/0297377, 2010/0295213, 2010/0295206, 2010/0028621, and 2006/0278355.

In a multi-ply dissolvable solid structure, one or more fibrous structure plies may be formed and/or deposited directly upon an existing ply of fibrous structure to form a multi-ply fibrous structure. The two or more existing fibrous structure plies may be combined, for example via thermal bonding, gluing, embossing, aperturing, rotary knife aperturing, die cutting, die punching, needle punching, knurling, pneumatic forming, hydraulic forming, laser cutting, tufting, and/or other mechanical combining process, with one or more other existing fibrous structure plies to form the multi-ply dissolvable solid structure described herein.

Pre-formed dissolvable fibrous web (comprised of dissolvable fibers and, optionally, agglomerate particles), having approximately ⅓ the total desired basis weight of the finished dissolvable solid structure, can be arranged in a face to face relationship with post-add minor ingredients disposed between layers, and laminated with a solid-state formation process. The resulting laminate is cut into the finished dissolvable solid structure shape via die cutting.

Lamination and Formation of Apertures Via Solid State Formation

The 3-layer web stack with minors disposed between layers can be passed together through a solid-state formation process (see Rotary Knife Aperturing apparatus below), forming roughly conical apertures in the dissolvable solid structure and causing inter-layer fiber interactions which result in a mechanically lamination dissolvable solid structure. Lamination aids (e.g. web plasticizing agents, adhesive fluids, etc.) may be additionally used to aid in secure lamination of layers.

Rotary Knife Aperturing Apparatus

Suitable solid-state description in disclosed in U.S. Pat. No. 8,679,391. Also, suitable dissolvable web aperturing description is disclosed in US 2016/0101026A1.

The nip comprises (2) intermeshed 100 pitch toothed rolls. The teeth on the toothed rolls have a pyramidal shape tip with six sides that taper from the base section of the tooth to a sharp point at the tip. The base section of the tooth has vertical leading and trailing edges and is joined to the pyramidal shape tip and the surface of the toothed roller. The teeth are oriented so the long direction runs in the MD.

The teeth are arranged in a staggered pattern, with a CD pitch P of 0.100 inch (2.5 mm) and a uniform tip to tip spacing in the MD of 0.223 inch (5.7 mm). The overall tooth height TH (including pyramidal and vertical base sections) is 0.270 inch (6.9 mm), the side wall angle on the long side of the tooth is 6.8 degrees and the side wall angle of the leading and trailing edges of the teeth in the pyramidal tip section is 25 degrees.

Opposing rollers are aligned such that the corresponding MD rows of each roller are in the same plane and such that the pins intermesh in a gear-like fashion with opposing pins passing near the center of the space between pins in the opposing roller MD row of pins. The degree of interference between the virtual cylinders described by the tips of the pins is described as the Depth of Engagement.

As web passes through the nip formed between the opposing rollers, the teeth from each roller engage with and penetrate the web to a depth determined largely by the depth of engagement between the rollers and the nominal thickness of the web.

### E. The Optional Preparing of the Surface Resident Coating Comprising the Active Agent

The preparation of the surface resident coating comprising the active agent may include any suitable mechanical, chemical, or otherwise means to produce a composition comprising the active agent(s) including any optional materials as described herein, or a coating from a fluid.

Optionally, the surface resident coating may comprise a water releasable matrix complex comprising active agent(s). The water releasable matrix complexes can comprise active agent(s) are prepared by spray drying wherein the active agent(s) is dispersed or emulsified within an aqueous composition comprising the dissolved matrix material under high shear (with optional emulsifying agents) and spray dried into a fine powder. The optional emulsifying agents can include gum arabic, specially modified starches, or other tensides as taught in the spray drying art (See Flavor Encapsulation, edited by Sara J. Risch and Gary A. Reineccius, pages 9, 45-54 (1988), which is incorporated herein by reference). Other known methods of manufacturing the water releasable matrix complexes comprising active agent(s) may include but are not limited to, fluid bed agglomeration, extrusion, cooling/crystallization methods and the use of phase transfer catalysts to promote interfacial polymerization. Alternatively, the active agent(s) can be adsorbed or absorbed into or combined with a water releasable matrix material that has been previously produced via a variety of mechanical mixing means (spray drying, paddle mixers, grinding, milling etc.). The water releasable matrix material in either pellet or granular or other solid-based form (and comprising any minor impurities as supplied by the supplier including residual solvents and plasticizers) may be ground or milled into a fine powder in the presence of the active agent(s) via a variety of mechanical means, for instance in a grinder or hammer mill.

Where the Dissolvable Solid Structure has a particulate coating, the particle size is known to have a direct effect on the potential reactive surface area of the active agents and thereby has a substantial effect on how fast the active agent delivers the intended beneficial effect upon dilution with water. In this sense, the active agents with smaller particle sizes tend to give a faster and shorter lived effect, whereas the active agents with larger particle sizes tend to give a slower and longer lived effect. The surface resident coatings may have a particle size from about 1 μm to about 200 μm, alternatively from about 2 μm to about 100 μm, and alternatively from about 3 μm to about 50 μm.

It can be helpful to include inert fillers within the grinding process, for instance aluminum starch octenylsuccinate under the trade name DRY-FLO® PC and available from Akzo Nobel, at a level sufficient to improve the flow properties of the powder and to mitigate inter-particle sticking or agglomeration during powder production or handling. Other optional excipients or cosmetic actives, as described herein, can be incorporated during or after the powder preparation process, e.g., grinding, milling, blending, spray drying, etc. The resulting powder may also be blended with other inert powders, either of inert materials or other powder-active complexes, and including water absorbing powders as described herein.

The active agents may be surface coated with non-hygroscopic solvents, anhydrous oils, and/or waxes as defined herein. This may include the steps of: (i) coating the water sensitive powder with the non-hydroscopic solvents, anhydrous oils, and/or waxes; (ii) reduction of the particle size of the active agent particulates, prior to, during, or after a coating is applied, by known mechanical means to a predetermined size or selected distribution of sizes; and (iii) blending the resulting coated particulates with other optional ingredients in particulate form. Alternatively, the coating of the non-hydroscopic solvents, anhydrous oils and/or waxes may be simultaneously applied to the other optional ingredients, in addition to the active agents, of the surface resident coating composition and with subsequent particle size reduction as per the procedure described above.

Where the coating is applied to the Structure as a fluid (such as by as a spray, a gel, or a cream coating), the fluid can be prepared prior to application onto the Structure or the fluid ingredients can be separately applied onto the Structure such as by two or more spray feed steams spraying separate components of the fluid onto the Structure.

Post-add minor ingredients can be applied to the surface of one or more web layers in the dissolvable solid structure, typically an interior surface. Individual minor ingredients may be applied together to a single selected surface or to separate surfaces. Minor ingredients may be applied to interior or exterior surfaces. In the present examples, minors were applied to the same interior surface, namely to one side of the middle of three layers.

Post-add ingredients in the present examples include fragrance and amodimethicone, both fluid at room temperature. Additional minor ingredients could include alternative conditioning agents, co-surfactants, encapsulated fragrance vehicles, rheology modifiers, etc. Minor ingredients could include fluids, particulates, pastes, or combinations.

In the present examples, fragrance is applied by atomizing through a spray nozzle (example Nordson EFD spray nozzle) and directing the resulting droplets of perfume to the target web surface, essentially uniformly over the surface.

In the present examples, amodimethicone is applied by expressing the fluid through an extrusion nozzle (example ITW-Dynatec UFD hot melt glue nozzle), comprising a series of orifices, approximately 500 microns in diameter and spaced at 2.5 mm, resulting in stripes of fluid extending the length of the target web surface.

Alternate fluid dispensing technologies, application patterns, and characteristic dimensions are contemplated.

### Methods of Use

The compositions described herein may be used for cleaning and/or treating hair, hair follicles, skin, teeth, and the oral cavity. The method for treating these consumer substrates may comprise the steps of: a) applying an effective amount of the Structure to the hand, b) wetting the Structure with water to dissolve the solid, c) applying the dissolved material to the target consumer substrate such as to clean or treat it, and d) rinsing the diluted treatment composition from the consumer substrate. These steps can be repeated as many times as desired to achieve the desired cleansing and or treatment benefit.

A method useful for providing a benefit to hair, hair follicles, skin, teeth, and/or the oral cavity, includes the step of applying a composition according to the first embodiment to these target consumer substrates in need of regulating.

The compositions can be used to condition hair. The conditioner can detangle, prevent breakage, increase hair strength, reduce frizz and/or static, help with styling, impart smoothness, softness, and/or shine, and/or repair damage. The conditioner can be applied as described herein. The conditioner can be used before, after, or simultaneously with a shampoo composition.

Alternatively a useful method for regulating the condition of hair, hair follicles, skin, teeth, the oral cavity, includes the step of applying one or more compositions described herein to these target consumer substrates in need of regulation.

The amount of the composition applied, the frequency of application and the period of use will vary widely depending upon the purpose of application, the level of components of a given composition and the level of regulation desired. For example, when the composition is applied for whole body or hair treatment, effective amounts generally range from about 0.5 grams to about 10 grams, alternatively from about 1.0 grams to about 5 grams, and alternatively from about 1.5 grams to about 3 grams.

### Product Types and Articles of Commerce

Non-limiting examples of products that utilize the dissolvable solid structures include hand cleansing substrates, teeth cleaning or treating substrates, oral cavity substrates, hair shampoo, conditioner, or other hair treatment substrates, body cleansing substrates, shaving preparation substrates, personal care substrates containing pharmaceutical or other skin care active, moisturizing substrates, sunscreen substrates, chronic skin benefit agent substrates (e.g., vitamin-containing substrates, alpha-hydroxy acid-containing substrates, etc.), deodorizing substrates, fragrance-containing substrates, and so forth.

Described herein is an article of commerce comprising one or more dissolvable solid structures described herein, and a communication directing a consumer to dissolve the Structure and apply the dissolved mixture to hair, hair follicles, skin, teeth, the oral cavity, to achieve a benefit to the target consumer substrate, a rapidly lathering foam, a rapidly rinsing foam, a clean rinsing foam, and combinations thereof. The communication may be printed material attached directly or indirectly to packaging that contains the dissolvable solid structure or on the dissolvable solid structure itself. Alternatively, the communication may be an electronic or a broadcast message that is associated with the article of manufacture. Alternatively, the communication may describe at least one possible use, capability, distinguishing feature and/or property of the article of manufacture.

### Test Methods

### Lamellar Structure Test Method

The Lamellar Structure Test Method makes use of small-angle x-ray scattering (SAXS) to determine if a lamellar structure is present in a dissolvable solid structure either in a conditioned, dry state or upon wetting after having been previously in a conditioned, dry state. Dissolvable solid structure are conditioned at a temperature of 23° C.±2.0° C. and a relative humidity of 40%±10% for a minimum of 12 hours prior to the test. Dissolvable solid structure conditioned as described herein are considered to be in a conditioned, dry state for the purposes of this invention. All instruments are calibrated according to manufacturer's specifications.

### Dry Sample Preparation

To prepare a sample to be analyzed directly in the conditioned, dry state, a specimen of about 1.0 cm diameter disc is isolated from the center of a dissolvable solid structure and is loaded into a conventional SAXS solid sample holder with aperture diameter between 4 and 5 mm. (Multiple specimen discs may be extracted from multiple dissolvable solid structures and stacked, if necessary, to ensure sufficient scattering cross-section.) The loaded sample holder is immediately placed in the appropriate instrument for data collection.

### Wet Sample Preparation

Three samples are analyzed upon wetting from the dry, conditioned state. Specimens are extracted from dry, conditioned dissolvable solid structure and hydrated with water in order to achieve three separate preparations each possessing a different material-to-water mass ratio. The three different material-to-water mass ratios to be prepared are 1:5; 1:9; and 1:20. For each mass ratio, one or more specimens (as needed) 1 cm in diameter are extracted from the geometric centers of one or more dissolvable solid structure in the dry, conditioned state are hydrated with 23° C.±2.0° C. filtered deionized (DI) water in order to achieve the intended material-to-water mass ratio. Each of the three material/water mixtures (each corresponding to a different mass ratio) is stirred under low shear gently by hand at room temperature using a spatula until visibly homogenous. Each material/water mixture is then immediately loaded into a separate quartz capillary tube with outer diameter 2.0 mm in diameter and 0.01 mm wall thickness. The capillary tubes are immediately sealed with a sealant such as an epoxy resin to prevent the evaporation of water from the preparations. The sealant is permitted to dry for at least 2 hours and until dry at a temperature of 23° C.±2.0° C. prior to sample analysis. Each prepared wet sample is introduced into an appropriate SAXS instrument and data are collected.

### Testing and Analysis

Samples are tested using SAXS in 2-dimension (2D) transmission mode over an angular range in of 0.3° to 3.0° 2θ, to observe the presence and spacing of any intensity bands in the x-ray scatter pattern. The test is conducted using a SAXS instrument (such as the NanoSTAR, Bruker AXS Inc., Madison, Wis., U.S.A., or equivalent). Conditioned, dry samples are analyzed under ambient pressure. Sealed liquid samples are analyzed in the instrument under vacuum. All samples are analyzed at a temperature of 23° C.±2.0° C. The x-ray tube of the instrument is operated sufficient power to ensure that any scattering bands present are clearly detected. The beam diameter is 550±50 μm. One suitable set of operating conditions includes the following selections: NanoSTAR instrument; micro-focus Cu x-ray tube; 45 kV and 0.650 mA power; Vantec2K 2-Dimensional area detector; collection time of 1200 seconds; and distance between the sample and detector of 112.050 cm. The raw 2-D SAXS scattering pattern is integrated azimuthally to determine intensity (I) as a function of the scattering vector (q), which are expressed throughout this method units of reciprocal angstroms (Å⁻¹). The values for q are calculated by the SAXS instrument according to the following equation:

$$q = \frac{4 π}{λ} sin θ$$

where:

2θ is the scattering angle; and

λ is the wavelength used.

For each integrated SAXS analyzed, the value of q in Å⁻¹ corresponding to each intensity peak on the plot of I vs q is identified and recorded from smallest to largest. (One of skill in the art knows that a sharp peak in q near the origin corresponds to scatter off of the beam stop and is disregarded in this method.) The value of q corresponding to the first intensity peak (the lowest value of q) is referred to as q*.

For a sample analyzed directly in the dry, conditioned state, if an intensity peak is present at 2q*±0.002 Å⁻¹, the sample is determined to exhibit a lamellar structure, and the characteristic d-spacing parameter is defined as 2π/q*. If no intensity peak if present at 2q*±0.002 Å⁻¹, the sample analyzed directly in the dry, conditioned state is determined to not exhibit a lamellar structure.

For a sample analyzed upon wetting from the dry, conditioned state, if an intensity peak is present at 2q*±0.002 Å⁻¹, the sample is determined to exhibit a lamellar structure, and the characteristic d-spacing parameter is defined as 2π/q*. If no intensity peak is present at 2q*±0.002 Å⁻¹, the sample is determined to not exhibit a lamellar structure. If a lamellar structure is determined to be present in at least any one of the three material/water ratios prepared, then this material is determined to exhibit a lamellar structure upon wetting. If no intensity peak is present at 2q*±0.002 Å⁻¹, in any of the three material/water ratios prepared, the material is determined to not exhibit a lamellar structure upon wetting.

Method for % Mass Loss measured at 96 hours

Place 500 g agglomerated particles in plastic bag with end open. Place plastic bag with Agglomerated Particle in beaker with open end up exposed to atmosphere. Allow to stand open for 96 hours. Weigh Agglomerated Particle. Calculate % loss.

### Basis Weight Measurement

In general, basis weight of a material or article (including the dissolvable solid structure) is measured by first cutting the sample to a known area, using a die cutter or equivalent, then measuring & recording the weight of the sample on a top-loading balance with a minimum resolution of 0.01 g, then finally by calculating the basis weight as follows:

Basis Weight (g/m2)=weight of basis weight pad (g)

$$Basis Weight ( \frac{g}{{m}^{2}} ) = \frac{Weight of pad ( g ) \times 10 , 000 \frac{{cm}^{2}}{{m}^{2}}}{area of pad ( {cm}^{2} )}$$

Suitable pad sample sizes for basis weight determination are >10 cm2 and should be cut with a precision die cutter having the desired geometry. If the dissolvable solid structure to be measured is smaller than 10 cm2, a smaller sampling area can be sued for basis weight determination with the appropriate changes to calculation.

In the present examples, basis weight was calculated based on the full dissolvable solid structure having a known area of 17.28 cm2. Thus, the basis weight calculation becomes:

$$Basis Weight ( \frac{g}{{m}^{2}} ) = \frac{Weight of pad ( g ) \times 10 , 000 \frac{{cm}^{2}}{{m}^{2}}}{17.28 {cm}^{2}}$$

### Thickness (Caliper) Measurement

The present examples were measured using the Check-Line J-40-V Digital Material Thickness Gauge from Electromatic Equipment Co. (Cedarhurst, N.Y.).

The sample (such as the dissolvable solid structure) is placed between a top and bottom plate of the instrument which has a top plate designed to apply a pressure of 0.5 kPa over a 25 cm2 area. The distance between the plates, to the nearest 0.01 mm, at the time of measurement is recorded as the thickness of the sample. The time of measurement is determined as the time at which the thickness in mm stabilizes or 5 seconds, whichever occurs first.

Equivalent methods are described in detail in compendial method ISO 9073-2, Determination of thickness for nonwovens, or equivalent.

### Bulk Density (Density) Determination

Bulk Density is determined by calculation given a Thickness and Basis Weight of the sample (the solid dissolvable structure) (using methods as described above) according to the following:

$$Bulk Density ( \frac{g}{{cm}^{3}} ) = \frac{Basis Weight of the pad ( \frac{g}{{m}^{2}} )}{\begin{matrix}Thickness of the pad ( mm ) \times \\ 0.1 \frac{cm}{mm} \times 10 , 000 \frac{{cm}^{2}}{{m}^{2}}\end{matrix}}$$

### Method of Measuring the Footprint of a Dissolvable Solid Structure (or Article)

The footprint of the dissolvable solid structure can be measured by measuring the dimensions of its base so that the base area (that is, the footprint) can be calculated. For example, in the case in which the base of the article is a parallelogram having right angles, the length of the unequal sides of the base (A and B) are measured by a ruler and the area of the base (footprint) is calculated as the product A×B. In the case in which the base of the dissolvable solid structure is a square, the length of a side (C) is measured by a ruler and the area of the base (footprint) is calculated as the square C2. Other examples of shapes can include circle, oval, etc.

### Hand Dissolution Test Method

### Materials Needed:

Dissolvable solid structures to be tested: 3-5 dissolvable solid structure s (finished product samples) are tested so that an average of the number of strokes for each if the individual dissolvable solid structure samples is calculated and recorded as the Average Hand Dissolution value for the dissolvable solid structure. For this method, the entire consumer saleable or consumer use dissolvable solid structure is tested. If the entire consumer saleable or consumer use dissolvable solid structure has a footprint greater than 50 cm², then first cut the dissolvable solid structure to have a footprint of 50 cm².

### Nitrile Gloves

10 cc syringe

Plastic Weigh boat (˜3 in×3 in)

100 mL Glass beaker

Water (City of Cincinnati Water or equivalent having the following properties: Total Hardness=155 mg/L as CaCO2; Calcium content=33.2 mg/L; Magnesium content=17.5 mg/L; Phosphate content=0.0462 mg/L)

Water used is 7 gpg hardness and 40° C.+/−5° C.

### Protocol:

Add 80 mL of water to glass beaker. Add 300-500 ml of water to glass beaker.
Heat water in beaker until water is at a temperature of 40° C.+/−5° C.
Transfer 10 mL of the water from the beaker into the weigh boat via the syringe.
Within 10 seconds of transferring the water to the weigh boat, place dissolvable solid structure sample in palm of gloved hand (hand in cupped position in non-dominant hand to hold dissolvable solid structure sample).
Using dominant hand, add water quickly from the weigh boat to the dissolvable solid structure sample and allow to immediately wet for a period of 5-10 seconds.
Rub with opposite dominant hand (also gloved) in 2 rapid circular strokes.
Visually examine the dissolvable solid structure sample in hand after the 2 strokes. If dissolvable solid structure sample is completely dissolved, record number of strokes=2 Dissolution Strokes. If not completely dissolved, rub remaining dissolvable solid structure sample for 2 more circular strokes (4 total) and observe degree of dissolution. If the dissolvable solid structure sample contains no solid pieces after the 2 additional strokes, record number of strokes=4 Dissolution Strokes. If after the 4 strokes total, the dissolvable solid structure sample still contains solid pieces of un-dissolved dissolvable solid structure sample, continue rubbing remaining dissolvable solid structure sample in additional 2 circular strokes and check if there are any remaining solid pieces of dissolvable solid structure sample after each additional 2 strokes until dissolvable solid structure sample is completely dissolved or until reaching a total of 30 strokes, whichever comes first. Record the total number of strokes. Record 30 Dissolution Strokes even if solid dissolvable solid structure sample pieces remain after the maximum of 30 strokes.
Repeat this process for each of the additional 4 dissolvable solid structure samples.
Calculate the arithmetic mean of the recorded values of Dissolution Strokes for the 5 individual dissolvable solid structure samples and record as the Average Hand Dissolution Value for the dissolvable solid structure. The Average Hand Dissolution Value is reported to the nearest single Dissolution Stroke unit.

### Fibrous Structures—Fiber Diameter

For fibrous Structures, the diameter of dissolvable fibers in a sample of a web is determined by using a Scanning Electron Microscope (SEM) or an Optical Microscope and image analysis software. A magnification of 200 to 10,000 times is chosen such that the fibers are suitably enlarged for measurement. When using the SEM, the samples are sputtered with gold or a palladium compound to avoid electric charging and vibrations of the fibers in the electron beam. A manual procedure for determining the fiber diameters is used from the image (on monitor screen) taken with the SEM or the optical microscope. Using a mouse and a cursor tool, the edge of a randomly selected fiber is sought and then measured across its width (i.e., perpendicular to fiber direction at that point) to the other edge of the fiber. A scaled and calibrated image analysis tool provides the scaling to get actual reading in microns (μm). Several fibers are thus randomly selected across the sample of the web using the SEM or the optical microscope. At least two specimens from the web (or web inside a product) are cut and tested in this manner. Altogether at least 100 such measurements are made and then all data are recorded for statistical analysis. The recorded data are used to calculate average (mean) of the fiber diameters, standard deviation of the fiber diameters, and median of the fiber diameters. Another useful statistic is the calculation of the amount of the population of fibers that is below a certain upper limit. To determine this statistic, the software is programmed to count how many results of the fiber diameters are below an upper limit and that count (divided by total number of data and multiplied by 100%) is reported in percent as percent below the upper limit, such as percent below 1 micron diameter or %-submicron, for example. We denote the measured diameter (in microns) of an individual circular fiber as di.

In case the fibers have non-circular cross-sections, the measurement of the fiber diameter is determined as and set equal to the hydraulic diameter which is four times the cross-sectional area of the fiber divided by the perimeter of the cross of the fiber (outer perimeter in case of hollow fibers). The number-average diameter, alternatively average diameter is calculated as, dₙᵤₘ

$$\frac{{\sum }_{i = 1}^{n} {d}_{i}}{n}$$

### Microscopic Detection Method

As used herein, “microscopic detection” means that a human with normal vision or vision that is corrected to normal vision (as used herein “normal vision” means that at 6 meters or 20 feet, a human eye with that performance is able to separate contours that are approximately 1.75 mm apart) the viewer can visually discern the quality of the example using a polarized light microscope with rotatable sample stage and at 100× magnification.

The microscope slide is prepared as follows, first small assemblies of fibers are manually teased from several locations of the fibrous structure using a tweezers such as Dumont style #3. These fibers are placed onto a glass microscope slide. One to two drops of immersion oil such as Zeiss Immersol 518 N (ISO 8036-1, ne=1.518) is used to improve the image quality of the fibers. The sample is covered with a cover slip. The fibers are imaged using an inverted light microscope with digital camera such as the Nikon Ti-U Manual Inverted Microscope with Zyla sCMOS camera with 100 W halogen illuminator. Images are taken in transmission bright field mode with and/or without cross-polars using a total magnification of about 100× to 200×. For 100× magnification a 10× objective lens such as a CFI Plan Fluor 10× Objective Lens (NA 0.3 WD 16 MM) can be used. Optimization of the images such as brightness, focus, and contrast is achieved by choosing the ideal settings for the camera, condenser and focus levels by one skilled in the art.

With crossed polars, if you rotate the filament 360°, using transmitted bright field lighting and do not see any discrete particles or globules, then the filament is homogeneous. However, if you rotate the filament 360° and if at any one direction you see discrete particles or globules, then the filament is heterogeneous.

### Non-Limiting Examples

The compositions illustrated in the following Examples illustrate specific embodiments of the composition, but are not intended to be limiting thereof. Other modifications can be undertaken by the skilled artisan without departing from the spirit and scope of this invention. These exemplified embodiments of the composition as described herein provide enhanced conditioning benefits to the hair.

All exemplified amounts are listed as weight percents and exclude minor materials such as diluents, preservatives, color solutions, imagery ingredients, botanicals, and so forth, unless otherwise specified. All percentages are based on weight unless otherwise specified.

### Conditioner Examples

The following conditioner examples can be as follows.

### Gel Network Premix Preparation

First, the gel network premix was prepared by heating stearyl alcohol, 1-Hexadeconol and the cationic surfactant(s) (e.g., behentrimonium methosulfate). The materials were heated to above the melt temperature of each material, about 85° C. Once the mixture was fully melted, water, glycerin and any acid were heated to 35-60° C. and then slowly added to the stearyl alcohol/1-hexadeconol/cation surfactant mixture with agitation. The mixture is then cooled below 35° C. During this cooling step, the fatty alcohols and cationic surfactant crystallize to form a crystalline gel network. Salt (e.g. sodium chloride) was then added with agitation. The Gel Network Premix for Examples 1-14, described hereafter, are in Table 1 and Table 2, below.

TABLE 1
Gel Network Premix for Fibrous Structures in Examples 1-7 in Table 3, respectively

| | Ex 1 | Ex 2 | Ex 3 | Ex 4 | Ex 5 | Ex 6 | Ex 7 |
|---|---|---|---|---|---|---|---|
| Water | 55.98 | 56.23 | 56.9 | 56.9 | 55.95 | 55.95 | 57.7 |
| Glycerin | 12 | 0 | 0 | 0 | 12 | 12 | 6 |
| Behentrimonium | 3.4 | 4.65 | 4.98 | 4.98 | 3.4 | 3.4 | 4.2 |
| Methosulfate¹ | | | | | | | |
| Stearamidopropyl- | 3.62 | 4.95 | 3.74 | 3.74 | 3.62 | 3.62 | 3.15 |
| dimethylamine² | | | | | | | |
| Stearyl Alcohol | 12.67 | 17.32 | 17.42 | 17.42 | 12.67 | 12.67 | 14.67 |
| 1-Hexadecanol | 6.33 | 8.65 | 8.71 | 8.71 | 6.33 | 6.33 | 7.33 |
| Sodium Chloride | 6 | 8.2 | 8.25 | 8.25 | 6 | 6 | 6.95 |
| Citric Acid | 0.00 | 0.00 | 0.00 | 0.00 | 0.03 | 0.03 | 0.00 |

¹Behentrimonium Methosulfate—IPA from Croda
²Stearamidopropyldimethylamine from Croda

TABLE 2
Gel Network Premix for Fibrous Structures in Examples 8-14 in Table 4, respectively

| | Ex 8 | Ex 9 | Ex 10 | Ex 11 | Ex 12 | Ex 13 | Ex 14 |
|---|---|---|---|---|---|---|---|
| Water | 55.95 | 56.2 | 68 | 70.5 | 56.2 | 60.24 | 60.21 |
| Glycerin | 12 | 0 | 0 | 0 | 0 | 0 | 0 |
| Behentrimonium | 3.4 | 4.65 | 8.55 | 8 | 4.65 | 8.89 | 0 |
| Methosulfate¹ | | | | | | | |
| Stearamidopropyl- | 3.62 | 4.95 | 0 | 0 | 4.95 | 0 | 8.89 |
| dimethylamine² | | | | | | | |
| Stearyl Alcohol | 12.67 | 17.32 | 13.24 | 12.3 | 17.32 | 16.84 | 16.84 |
| 1-Hexadecanol | 6.33 | 8.65 | 5.3 | 5 | 8.65 | 8.42 | 8.42 |
| Sodium Chloride | 6 | 8.2 | 2 | 1.5 | 8.2 | 5.61 | 5.61 |
| Dicetyldimonium | 0.00 | 0.00 | 2.91 | 2.70 | 0.00 | 0.00 | 0.00 |
| Chloride³ | | | | | | | |
| Citric Acid | 0.03 | 0.00 | 0.00 | 0.00 | 0.00 | 0.00 | 0.03 |

¹Behentrimonium Methosulfate—IPA from Croda
²Stearamidopropyldimethylamine from Croda
³Varisoft ® 432PPG from Evonik

### Filament-Forming Material Preparation

Separately, water was heated to above 75° C. Polyvinyl alcohol was added with agitation and mixed until completely dissolved. Other cationic or nonionic polymers were added under agitation and cooled to below 35° C. Then, the gel network premix was added with agitation along with any remaining salt and water to form the filament-forming material.

Next, the filament-forming material was spun into one or more filaments by meltblowing, as described herein. The filaments were collected and formed into fibrous structures. Finally, post-add minor ingredients, including fragrance and amodimethicone, were applied to the surface of one or more web layers of the fibrous structure. The fragrance and amodimenticone were typically applied to an interior surface.

The post-add minor ingredients can be applied to the structure as a fluid (such as by as a spray, a gel, or a cream coating), the fluid can be prepared prior to application onto the structure or the fluid ingredients can be separately applied onto the structure such as by two or more spray feed steams spraying separate components of the fluid onto the structure. Individual minor ingredients may be applied together to a single selected surface or to separate surfaces. Minor ingredients may be applied to interior or exterior surfaces. In the present examples, minors were applied to the same interior surface, namely to one side of the middle of three layers.

Post-add ingredients in the present examples included fragrance and amodimethicone, both fluid at room temperature. Additional minor ingredients could include alternative conditioning agents, co-surfactants, encapsulated fragrance vehicles, rheology modifiers, etc. Minor ingredients could include fluids, particulates, pastes, or combinations.

Table 3 and Table 4 are examples of dissolvable solid structures and are in grams on a dry fibrous structure basis.

TABLE 3
Fibrous Structures

| | Ex 1 | Ex 2 | Ex 3 | Ex 4 | Ex 5 | Ex 6 | Ex 7 |
|---|---|---|---|---|---|---|---|
| Water | 3.96 | 3.58 | 3.44 | 3.48 | 3.47 | 3.47 | 3.47 |
| Guar, Hydroxylpropyl | 0.67 | 0.79 | 0.91 | 0.78 | 0.67 | 0.67 | 0.80 |
| Trimonium Chloride, | | | | | | | |
| Jaguar ® C-500¹ | | | | | | | |
| Glycerin | 14.17 | 0.00 | 2.39 | 2.06 | 14.25 | 14.25 | 7.95 |
| Behentrimonium | 2.66 | 3.28 | 3.85 | 3.32 | 2.68 | 2.68 | 3.38 |
| Methosulfate² | | | | | | | |
| Stearamidopropyl- | 3.54 | 4.37 | 3.61 | 3.12 | 3.56 | 3.56 | 3.17 |
| dimethylamine³ | | | | | | | |
| Stearyl Alcohol | 12.40 | 15.29 | 16.83 | 14.53 | 12.47 | 12.47 | 14.77 |
| 1-Hexadecanol | 6.20 | 7.64 | 8.41 | 7.26 | 6.23 | 6.23 | 7.38 |
| Sodium Chloride | 5.87 | 7.24 | 7.97 | 6.88 | 5.91 | 5.91 | 7.00 |
| Dicetyldimonium | 0.00 | 0.00 | 0.00 | 0.00 | 0.00 | 0.00 | 0.00 |
| Chloride⁴ | | | | | | | |
| Citric Acid | 0.00 | 0.00 | 0.00 | 0.00 | 0.03 | 0.03 | 0.00 |
| Polyvinyl Alcohol⁵ | 18.17 | 0.00 | 18.87 | 0.00 | 18.27 | 18.27 | 0.00 |
| Polyvinyl Alcohol⁶ | 18.17 | 0.00 | 18.87 | 0.00 | 18.27 | 18.27 | 0.00 |
| Polyvinyl Alcohol⁷ | 0.00 | 0.00 | 0.00 | 0.00 | 0.00 | 0.00 | 0.00 |
| Polyvinyl Alcohol⁸ | 0.00 | 43.70 | 0.00 | 44.50 | 0.00 | 0.00 | 38.06 |
| PEG45-M⁹ | 0.81 | 0.73 | 0.80 | 0.69 | 0.81 | 0.81 | 0.64 |
| Amodimethicone¹⁰ | 5.51 | 5.51 | 5.79 | 5.51 | 5.51 | 5.51 | 5.51 |
| Fragrance | 7.87 | 7.87 | 8.26 | 7.87 | 7.87 | 7.87 | 7.87 |
| Hand Dispersion Value | 30 | 25 | 30 | 25 | 30 | 30 | 25 |
| (strokes) | | | | | | | |
| Presence of Lamellar | Yes | Yes | Yes | Yes | Yes | Yes | Yes |
| Structure | | | | | | | |
| Heterogeneous Filaments | Yes | Yes | Yes | Yes | Yes | Yes | Yes |

¹Jaguar ® C500, MW of 500,000, CD of 0.7, from Solvay
²Behentrimonium Methosulfate—IPA from Croda
³Stearamidopropyl Dimethylamine from Croda
⁴Varisoft ® 432PPG from Evonik
⁵Kuraray Poval ™ 32-80 PVA420h from Kuraray
⁶Kuraray Poval ™ 3-80 PVA 403 from Kuraray
⁷Kuraray Poval ™ 5-74 PVA 505 from Kuraray
⁸Selvol ™ 205 from SCKISUI
⁹Polyox ™ WSR N60K from Amerchol
¹⁰Amodimethicone from Momentive ™ Performance Materials

TABLE 4
Fibrous Structures

| | Ex 8 | Ex 9 | Ex 10 | Ex 11 | Ex 12 | Ex 13 | Ex 14 |
|---|---|---|---|---|---|---|---|
| Water | 3.41 | 3.47 | 3.43 | 3.40 | 3.55 | 5.00 | 4.97 |
| Guar, Hydroxylpropyl | 0.66 | 0.80 | 0.00 | 0.00 | 0.94 | 0.00 | 0.00 |
| Trimonium Chloride, Jaguar ® | | | | | | | |
| C-500 | | | | | | | |
| Glycerin | 14.00 | 0.00 | 0.00 | 0.00 | 0.00 | 0.00 | 0.00 |
| Behentrimonium | 2.63 | 3.31 | 10.79 | 8.42 | 3.87 | 9.5 | 0.00 |
| Methosulfate² | | | | | | | |
| Stearamidopropyl- | 3.50 | 4.40 | 0.00 | 0.00 | 5.15 | 0.00 | 9.5 |
| dimethylamine³ | | | | | | | |
| Stearyl Alcohol | 12.26 | 15.39 | 20.89 | 16.18 | 18.02 | 18.00 | 18.00 |
| 1-Hexadecanol | 6.12 | 7.69 | 8.36 | 6.58 | 9.00 | 9.00 | 9.00 |
| Sodium Chloride | 5.80 | 7.29 | 3.16 | 1.97 | 8.53 | 6.0 | 6.0 |
| Dicetyldimonium Chloride⁴ | 0.00 | 0.00 | 4.59 | 3.55 | 0.00 | 0.00 | 0.00 |
| Citric Acid | 0.03 | 0.03 | 0.00 | 0.00 | 0.03 | 0.00 | 0.03 |
| Polyvinyl Alcohol⁵ | 17.96 | 0.00 | 9.96 | 13.13 | 15.46 | 18.5 | 18.5 |
| Polyvinyl Alcohol⁶ | 17.96 | 0.00 | 0.00 | 0.00 | 0.00 | 18.5 | 18.5 |
| Polyvinyl Alcohol⁷ | 0.00 | 0.00 | 23.25 | 30.63 | 0.00 | 0.00 | 0.00 |
| Polyvinyl Alcohol⁸ | 0.00 | 43.98 | 0.00 | 0.00 | 23.18 | 0.00 | 0.00 |
| PEG45-M⁹ | 0.80 | 0.73 | 1.44 | 1.44 | 0.86 | 1.00 | 1.00 |
| Amodimethicone¹⁰ | 6.12 | 5.32 | 5.82 | 6.05 | 4.70 | 6.00 | 6.00 |
| Fragrance | 8.75 | 7.59 | 8.31 | 8.65 | 6.71 | 8.50 | 8.50 |
| Hand Dispersion Value | 30 | 25 | 30 | 30 | 30 | 30 | 30 |
| (strokes) | | | | | | | |
| Presence of Lamellar Structure | Yes | Yes | Yes | Yes | Yes | Yes | Yes |
| Heterogeneous Filaments | Yes | Yes | Yes | Yes | Yes | Yes | Yes |

¹¹Jaguar ® C500, MW of 500,000, CD of 0.7, from Solvay
¹²Behentrimonium Methosulfate—IPA from Croda
¹³Stearamidopropyl Dimethylamine from Croda
¹⁴Varisoft ® 432PPG from Evonik
¹⁵Kuraray Poval ™ 32-80 PVA420h from Kuraray
¹⁶Kuraray Poval ™ 3-80 PVA 403 from Kuraray
¹⁷Kuraray Poval ™ 5-74 PVA 505 from Kuraray
¹⁸Selvol ™ 205 from SCKISUI
¹⁹Polyox ™ WSR N60K from Amerchol
²⁰Amodimethicone from Momentive ™ Performance Materials

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A dissolvable solid structure comprising filaments wherein the filaments comprise:
a. from about 30% to about 45%, by weight on a dry filament basis, of a water-soluble polymeric structurant;
b. from about 15% to about 27%, by weight on a dry filament basis, of a fatty alcohol having a carbon chain length from about 16 to about 22 carbon atoms or mixtures thereof and a melting point above 25° C.;
c. from about 5% to about 10%, by weight on a dry filament basis, cationic surfactant;
wherein the filament is heterogeneous upon microscopic inspection and comprises one or more globules comprising a dehydrated gel network wherein the dehydrated gel network comprises the fatty alcohol and the cationic surfactant.

2. The dissolvable solid structure of claim 1 wherein the composition comprises less than 7%, by weight on a dry filament basis, water.

3. The dissolvable solid structure of claim 1 wherein the water-soluble polymeric structurant is selected from the group consisting of polyethylene oxides, polyvinyl alcohols, polyacrylates, vinyl acetate-vinyl alcohol copolymer, copolymers of acrylic acid and methacrylic acid, polyvinylmethylether, polyvinylformamide, polyacrylamide, polyvinylpyrrolidones, starch and starch derivatives, pullulan, hydroxypropylmethylcelluloses, methylcelluloses, carboxymethycelluloses, salts and combinations thereof.

4. The dissolvable solid structure of claim 3 wherein the water-soluble polymeric structurant is selected from the group consisting of vinyl acetate-vinyl alcohol copolymer, polyvinyl alcohol, and combinations thereof.

5. The dissolvable solid structure of claim 1 wherein the fatty alcohol is selected from the group consisting of cetyl alcohol, stearyl alcohol, behenyl alcohol, and combinations thereof.

6. The dissolvable solid structure of claim 1 wherein the cationic surfactant is selected from the group consisting of mono-long alkyl quaternized ammonium salt, mono-long alkyl amine, and combinations thereof.

7. The dissolvable solid structure of claim 6 wherein the cationic surfactant comprises a mono-long alkyl quaternized ammonium salt selected from the group consisting of behenyl trimethyl ammonium salt; stearyl trimethyl ammonium salt; cetyl trimethyl ammonium salt; and hydrogenated tallow alkyl trimethyl ammonium salt, and combinations thereof.

8. The dissolvable solid structure of claim 7 wherein the cationic surfactant comprises behenyl trimethyl ammonium salt.

9. The dissolvable solid structure of claim 1 wherein the filament comprises a diameter of less than 25 microns and the nodule comprises a width that is less than the diameter.

10. The dissolvable solid structure of claim 1 wherein the nodule comprises a width less than 20 microns.

11. The dissolvable solid structure of claim 1 wherein the dissolvable solid structure is a hair conditioner.

12. The dissolvable solid structure of claim 1 wherein the dissolvable structure comprises a lamellar structure upon addition of water to the dissolvable solid structure in the ratio of about 5:1.

13. A dissolvable solid structure comprising filaments wherein the filaments comprise:
a. from about 15% to about 55%, by weight on a dry filament basis, of a polymeric structurant;
b. a globule comprising a dehydrated gel network comprising a high melting point fatty material having a carbon chain length C12-C22 or mixtures thereof, wherein the melting point is above 25° C.; and a cationic surfactant;
wherein the filaments are heterogeneous upon microscopic inspection;
wherein the dissolvable structure comprises a lamellar structure upon addition of water to the dissolvable solid structure in the ratio of about 5:1.

14. The dissolvable solid structure of claim 13 wherein the dissolvable solid structure comprises the lamellar structure without the addition of water to the dissolvable solid structure.

15. The dissolvable solid structure of claim 13, further comprising from about 1% to about 30%, by weight on a dry filament basis, of a dispersing agent selected from the group consisting of a surfactant from the nonionic class of alkyl glucamides, reverse alkyl glucamides, cocoamiodpropyl betaines, alkyl glucoside, triethanol amine, cocamide MEAs and combinations thereof.

16. The dissolvable solid structure of claim 13, wherein the filament comprises from about 1% to about 15%, by weight on a dry filament basis, cationic surfactant wherein the cationic surfactant comprises a mono-long alkyl quaternized ammonium salt selected from the group consisting of behenyl trimethyl ammonium salt; stearyl trimethyl ammonium salt; cetyl trimethyl ammonium salt; and hydrogenated tallow alkyl trimethyl ammonium salt, and combinations thereof.

17. The dissolvable solid structure of claim 13, wherein the high melting point fatty material is selected from the group consisting of a fatty alcohol and a blend of one or more fatty alcohols.

18. The dissolvable solid structure of claim 13, further comprising from about 0.1% to about 5%, by weight on a dry filament basis, of a volatile solvent selected from the group consisting of water, acetic acid, acetone methanol, ethanol, propanol, isopropyl alcohol, butanol, methylethyl ketones, and combinations thereof.

19. The structure of claim 2, wherein the Dissolvable Solid Structure dissolves in less than about 30 strokes of the Hand Dissolution Method.

20. A dissolvable solid structure comprising filaments wherein the filaments comprise:
a. from about 30% to about 45%, by weight on a dry filament basis, of a water-soluble polymer selected from the group consisting of polyvinyl alcohol, vinyl acetate-vinyl alcohol copolymer, and combinations thereof;
b. from about 15% to about 27%, by weight on a dry filament basis, of a fatty alcohol selected from the group consisting of fatty alcohol is selected from the group consisting of cetyl alcohol, stearyl alcohol, behenyl alcohol, and combinations thereof;
c. from about 5% to about 10%, by weight on a dry filament basis, of a mono-long alkyl quaternized ammonium salt selected from the group consisting of behenyl trimethyl ammonium salt; stearyl trimethyl ammonium salt; cetyl trimethyl ammonium salt; and hydrogenated tallow alkyl trimethyl ammonium salt, and combinations thereof;
wherein the filament is heterogeneous upon microscopic inspection and comprises one or more globules comprising a dehydrated gel network wherein the dehydrated gel network comprises the fatty alcohol and the cationic surfactant;
wherein the dissolvable solid structure is a hair conditioner.
